(19) United States
(12) Patent Application Publication
(10) Pub. No.: US 2021/0261524 A1
(43) Pub. Date: Aug. 26, 2021

(54) THERAPEUTIC COMPOUNDS AND COMPOSITIONS

(71) Applicant: Exithera Pharmaceuticals, Inc., Wesborough, MA (US)

(72) Inventors: Neil J. HAYWARD, Westborough, MA (US); Bertrand L. CHENARD, Waterford, CT (US); Yuelian XU, East Haven, CT (US); Roberta L. DOROW, Kalamazoo, MI (US); Michael E. MATISON, Middleville, MI (US); Alexander KOLCHINSKI, Winchester, MA (US); Richard FORNICOLA, Littleton, MA (US)

(21) Appl. No.: 17/243,120

(22) Filed: Apr. 28, 2021

Related U.S. Application Data
(63) Continuation of application No. PCT/US2019/058896, filed on Oct. 30, 2019.
(60) Provisional application No. 62/752,503, filed on Oct. 30, 2018.

(51) Int. Cl. C07D 401/06; A61K 9/00; A61P 7/02; A61M 1/36; A61L 33/00; A61L 33/04

(52) U.S. Cl. CPC C07D 401/06; A61K 9/0019; A61P 7/02; C07B 2200/13; A61L 33/0041; A61L 33/04; A61M 1/3673

(57) ABSTRACT

The present invention provides compounds that inhibit Factor XIa or kallikrein and pharmaceutically acceptable salts thereof and compositions thereof. The present invention also provides methods of making these compounds or pharmaceutically acceptable salts thereof and compositions and methods of use thereof.

### CROSS-REFERENCE TO RELATED APPLICATIONS

This application is a continuation of International Application No. PCT/US2019/058896 filed Oct. 30, 2019, which claims the benefit and priority to U.S. Ser. No. 62/752,503 filed Oct. 30, 2018, each of which is incorporated herein by reference in its entirety.

### BACKGROUND OF THE INVENTION

Blood coagulation is the first line of defense against blood loss following injury. The blood coagulation “cascade” involves a number of circulating serine protease zymogens, regulatory cofactors and inhibitors. Each enzyme, once generated from its zymogen, specifically cleaves the next zymogen in the cascade to produce an active protease. This process is repeated until finally thrombin cleaves the fibrinopeptides from fibrinogen to produce fibrin that polymerizes to form a blood clot. Although efficient clotting limits the loss of blood at a site of trauma, it also poses the risk of systemic coagulation resulting in massive thrombosis. Under normal circumstances, hemostasis maintains a balance between clot formation (coagulation) and clot dissolution (fibrinolysis). However, in certain disease states such as acute myocardial infarction and unstable angina, the rupture of an established atherosclerotic plaque results in abnormal thrombus formation in the coronary arterial vasculature.

Diseases that stem from blood coagulation, such as myocardial infarction, unstable angina, atrial fibrillation, stroke, pulmonary embolism, and deep vein thrombosis, are among the leading causes of death in developed countries. Current anticoagulant therapies, such as injectable unfractionated and low molecular weight (LMW) heparin and orally administered warfarin (coumadin), carry the risk of bleeding episodes and display patient-to-patient variability that results in the need for close monitoring and titration of therapeutic doses. Consequently, there is a large medical need for novel anticoagulation drugs that lack some or all of the side effects of currently available drugs.

Factor XIa is an attractive therapeutic target involved in the pathway associated with these diseases. Increased levels of Factor XIa or Factor XIa activity have been observed in several thromboembolic disorders, including venous thrombosis (Meijers et al., N. Engl. J. Med. 342:696, 2000), acute myocardial infarction (Minnema et al., Arterioscler Thromb Vasc Biol 20:2489, 2000), acute coronary syndrome (Butenas et al., Thromb Haemost 99:142, 2008), coronary artery disease (Butenas et al., Thromb Haemost 99:142, 2008), chronic obstructive pulmonary disease (Jankowski et al., Thromb Res 127:242, 2011), aortic stenosis (Blood Coagul Fibrinolysis, 22:473, 2011), acute cerebrovascular ischemia (Undas et al., Eur J Clin Invest, 42:123, 2012), and systolic heart failure due to ischemic cardiomyopathy (Zabcyk et al., Pol Arch Med Wewn. 120:334, 2010). Patients that lack Factor XI because of a genetic Factor XI deficiency exhibit few, if any, ischemic strokes (Salomon et al., Blood, 111:4113, 2008). At the same time, loss of Factor XIa activity, which leaves one of the pathways that initiate coagulation intact, does not disrupt hemostasis. In humans, Factor XI deficiency can result in a mild-to-moderate bleeding disorder, especially in tissues with high levels of local fibrinolytic activity, such as the urinary tract, nose, oral cavity, and tonsils. Moreover, hemostasis is nearly normal in Factor XI-deficient mice (Gailani, Blood Coagul Fibrinolysis, 8:134, 1997). Furthermore, inhibition of Factor XI has also been found to attenuate arterial hypertension and other diseases and dysfunctions, including vascular inflammation (Kossmann et al. Sci. Transl. Med. 9, eaah4923 (2017)).

Consequently, compounds that inhibit Factor XIa have the potential to prevent or treat a wide range of disorders while avoiding the side effects and therapeutic challenges that plague drugs that inhibit other components of the coagulation pathway. Moreover, due to the limited efficacy and adverse side effects of some current therapeutics for the inhibition of undesirable thrombosis (e.g., deep vein thrombosis, hepatic vein thrombosis, and stroke), improved compounds and methods (e.g., those associated with Factor XIa) are needed for preventing or treating undesirable thrombosis.

Another therapeutic target is the enzyme kallikrein. Human plasma kallikrein is a serine protease that may be responsible for activating several downstream factors (e.g., bradykinin and plasmin) that are critical for coagulation and control of e.g., blood pressure, inflammation, and pain. Kallikreins are expressed e.g., in the prostate, epidermis, and the central nervous system (CNS) and may participate in e.g., the regulation of semen liquefaction, cleavage of cellular adhesion proteins, and neuronal plasticity in the CNS. Moreover, kallikreins may be involved in tumorigenesis and the development of cancer and angioedema, e.g., hereditary angioedema. Overactivation of the kallikrein-kinin pathway can result in a number of disorders, including angioedema, e.g., hereditary angioedema (Schneider et al., J. Allergy Clin. Immunol. 120:2, 416, 2007). To date, there are limited treatment options for HAE (e.g., WO2003/076458). As such, therapeutics are needed for preventing or treating these diseases.

### SUMMARY OF THE INVENTION

The present invention features compounds that inhibit Factor XIa or kallikrein and methods for preventing or treating undesired thrombosis or angiodema (e.g., hereditary angiodema) by administering one or more of these compounds alone or in combination with other molecules to a mammal. The invention also provides methods for designing or selecting additional Factor XIa or kallikrein inhibitors using these structures. Desirably, these compounds have certain structural, physical, and spatial characteristics that enable the compounds to interact with specific residues of the active site of Factor XIa or kallikrein.

In one aspect, the present invention is directed to Compound 1:

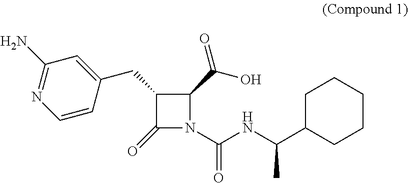 [SMILES: C[C@@H](NC(=O)N1C(=O)[C@H](Cc2ccnc(N)c2)[C@H]1C(=O)O)C1CCCCC1]

or a pharmaceutically acceptable salt thereof, e.g., a hydrochloride salt of Compound 1. In some embodiments, Compound 1 or a pharmaceutically acceptable salt thereof is crystalline. In some embodiments, Compound 1 or a pharmaceutically acceptable salt thereof exists as a substantially pure crystalline solid form.

In one aspect, provided herein is a pharmaceutically acceptable salt of Formula (I):

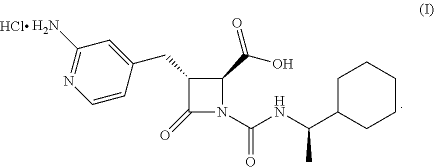 [SMILES: C[C@@H](NC(=O)N1C(=O)[C@H](Cc2ccnc(N)c2)[C@H]1C(=O)O)C1CCCCC1]

The pharmaceutically acceptable salt of Formula (I) is a hydrochloride salt of Compound 1 and also referred to herein as Compound 1.HCl. In some embodiments, Compound 1.HCl is crystalline. In some embodiments, Compound 1.HCl exists as a substantially pure crystalline solid form. In some embodiments, Compound 1.HCl has an XRPD pattern substantially as depicted in FIG. 9.

In one aspect, provided herein is a process for preparing a pharmaceutically acceptable salt of Formula (I):

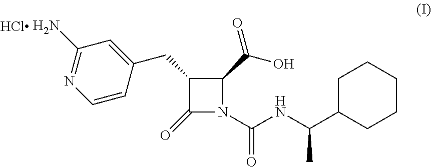 [SMILES: C[C@@H](NC(=O)N1C(=O)[C@H](Cc2ccnc(N)c2)[C@H]1C(=O)O)C1CCCCC1]

or a solvate (e.g., a hydrate) thereof, comprising dissolving a salt of Formula (II)

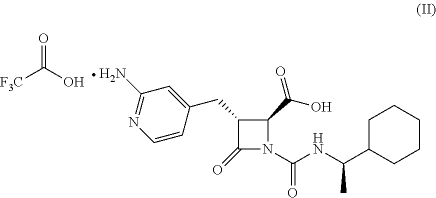 [SMILES: O=C(O)C(F)(F)F ; C[C@@H](NC(=O)N1C(=O)[C@H](Cc2ccnc(N)c2)[C@H]1C(=O)O)C1CCCCC1]

or a solvate (e.g., a hydrate) thereof in a solvent, thereby preparing a first solution, and adding hydrogen chloride to the first solution, thereby producing the pharmaceutically acceptable salt of Formula (I).

In some embodiments, the salt of Formula (II) is dissolved in an aprotic solvent. In some embodiments, the solvent comprises (e.g., consists of or consists essentially of) acetonitrile. In some embodiments, the hydrogen chloride is added to the first solution by bubbling HCl gas into the first solution or by adding a separate solution comprising HCl (e.g., an ethereal hydrochloric acid solution) to the first solution.

In some embodiments, the starting quantity of the salt of Formula (II) or solvate (e.g., a hydrate) thereof is greater than or equal to 500 grams. In some embodiments, the starting quantity of the salt of Formula (II) or solvate (e.g., a hydrate) thereof is greater than or equal to 1 kilogram. In some embodiments, the process produces over 300 grams (e.g., over about 350 grams (e.g., about 368 grams)) of pharmaceutically acceptable salt of Formula (I) or solvate (e.g., a hydrate) thereof.

In some embodiments, the process produces the pharmaceutically acceptable salt of Formula (I) or solvate (e.g., a hydrate) thereof in a yield greater than about 50% (e.g., in about 55% yield). In some embodiments, the process produces the pharmaceutically acceptable salt of Formula (I) or solvate (e.g., a hydrate) thereof in a yield greater than about 75%. In some embodiments, the process produces the pharmaceutically acceptable salt of Formula (I) or solvate (e.g., a hydrate) thereof in a yield greater than about 90%. In some embodiments, the process produces the pharmaceutically acceptable salt of Formula (I) or solvate (e.g., a hydrate) thereof in a yield greater than about 99%. In some embodiments, the purity of the pharmaceutically acceptable salt of Formula (I) or solvate (e.g., a hydrate) thereof is about 80%. In some embodiments, the purity of the pharmaceutically acceptable salt of Formula (I) or solvate (e.g., a hydrate) thereof is about 81%.

In some embodiments, the process further comprises purifying the pharmaceutically acceptable salt of Formula (I) or solvate (e.g., a hydrate) thereof by dissolving the pharmaceutically acceptable salt of Formula (I) or solvate (e.g., a hydrate) thereof in a solvent (e.g., isopropyl alcohol) followed by precipitation of the dissolved pharmaceutically acceptable salt of Formula (I) or solvate (e.g., a hydrate) thereof using another solvent (e.g., methyl tert-butyl ether). In some embodiments, the purity of the pharmaceutically acceptable salt of Formula (I) or solvate (e.g., a hydrate) thereof after precipitation is greater than 98%. In some embodiments, the purity of the pharmaceutically acceptable salt of Formula (I) or solvate (e.g., a hydrate) thereof after precipitation is about 98%.

In some embodiments, the process further comprises purifying the pharmaceutically acceptable salt of Formula (I) or solvate (e.g., a hydrate) thereof by slurrying the pharmaceutically acceptable salt of Formula (I) or solvate (e.g., a hydrate) thereof in a solvent (e.g., isopropyl alcohol) and then filtering the pharmaceutically acceptable salt of

Formula (I) or solvate (e.g., a hydrate) thereof to separate the pharmaceutically acceptable salt of Formula (I) or solvate (e.g., a hydrate) thereof from the solvent. In some embodiments, the purity of the pharmaceutically acceptable salt of Formula (I) or solvate (e.g., a hydrate) thereof after slurrying and separating is greater than 98%. In some embodiments, the purity of the pharmaceutically acceptable salt of Formula (I) or solvate (e.g., a hydrate) thereof after slurrying and separating is about 98%.

In some embodiments, the process comprises preparing the salt of Formula (II) by contacting a compound of Formula (III)

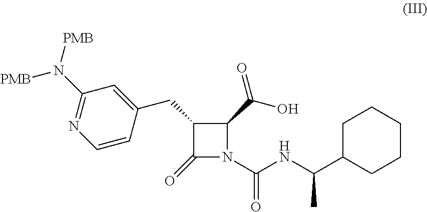

with trifluoroacetic acid.

In some embodiments, the process further comprises contacting the compound of Formula (III) with a silane (e.g., triethylsilane).

In some embodiments, the process produces over 500 grams of the compound of Formula (III) (e.g., over 1 kg).

In some embodiments, the process comprises preparing the compound of Formula (III) by contacting a compound of Formula (IV)

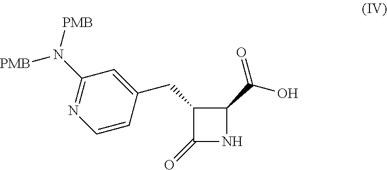

with a compound of Formula (V)

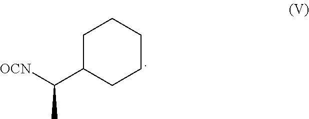

In some embodiments, process produces over 1 kilogram of the compound of Formula (III) (e.g., about 1.3 kg). In some embodiments, the process is carried out in the presence of a solvent. In some embodiments, the process is carried out in the presence of a base, (e.g., 1,8-diazabicyclo(5.4.0)undec-7-ene).

In some embodiments, the process comprises preparing a compound of Formula (IV) by contacting a compound of Formula (VI)

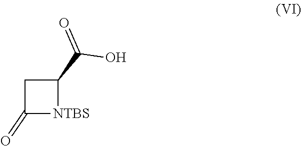

with the compound of Formula (VII)

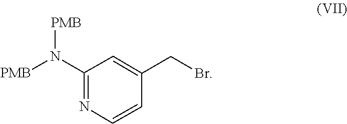

In some embodiments, the process produces over 500 grams of the compound of Formula (IV) (e.g., over 900 grams).

In some embodiments, the compound of Formula (III) is purified by a purification method that is not chromatography. In some embodiments, the purification method comprises slurrying the compound of Formula (III) in a solvent (e.g., heptane) and then filtering the compound of Formula (III) to separate the compound of Formula (III) from the solvent. In some embodiments, the purity of the compound of Formula (III) is greater than 90%.

In some embodiments, the compound of Formula (I) is purified by a purification method that is not chromatography.

In one aspect, provided herein is a crystalline pharmaceutically acceptable salt of the Formula (I):

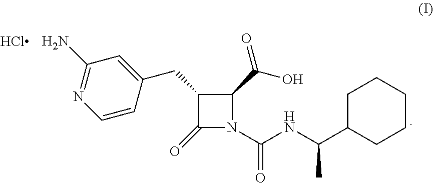 [SMILES: C[C@@H](NC(=O)N1C(=O)[C@H](Cc2ccnc(N)c2)[C@H]1C(=O)O)C1CCCCC1]

In one aspect, the present invention is directed to a pharmaceutical composition comprising Compound 1 or a pharmaceutically acceptable salt thereof and one or more pharmaceutically acceptable excipients. In some embodiments, the composition is provided as a liquid formulation (e.g., a solution). In some embodiments, the composition is provided as a solid formulation (e.g., a capsule, pill, tablet, or powder).

In some embodiments, each crystalline solid form is characterized and identified with parameters obtained from one or more of the aforementioned analytical methods:

percent API (free base) and estimated purity of each sample as determined by HPLC analysis; X-ray diffraction patterns are presented with degrees 2-theta (2θ) as the abscissa and peak intensity as the ordinate as determined by analysis with XRPD. These patterns are also referred to herein as XRPD patterns.

In some embodiments, a solid form is determined to be crystalline by the presence of sharp, distinct peaks found in the corresponding XRPD pattern.

In an aspect, provided herein is a method of treating a thromboembolic disorder in a subject in need thereof, the method comprising administering to the subject an effective amount of a compound represented by

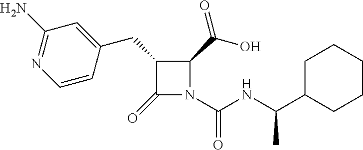 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In an aspect, provided herein is a method of reducing the risk of a thromboembolic disorder in a subject in need thereof, the method comprising administering to the subject an effective amount of a compound represented by

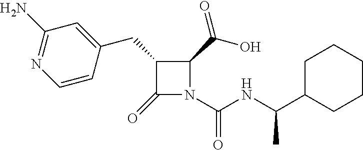 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In an aspect, provided herein is a method of prophylaxis of a thromboembolic disorder in a subject in need thereof, the method comprising administering to the subject an effective amount of a compound represented by

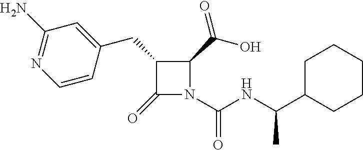 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In some embodiments of the methods provided herein, the artificial surface is in contact with blood in the subject's circulatory system. In some embodiments, the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus. In some embodiments, the artificial surface causes or is associated with the thromboembolic disorder. In some embodiments, the thromboembolic disorder is a venous thromboembolism, deep vein thrombosis, or pulmonary embolism. In some embodiments, the thromboembolic disorder is a blood clot.

In some embodiments, the methods further comprise conditioning the artificial surface with a separate dose of the compound or pharmaceutically acceptable salt thereof, prior to contacting the artificial surface with blood in the circulatory system of the subject. In some embodiments, the methods further comprise conditioning the artificial surface with a separate dose of the compound or pharmaceutically acceptable salt thereof prior to or during administration of the compound or a pharmaceutically acceptable salt thereof to the subject. In some embodiments, the methods further comprise conditioning the artificial surface with a separate dose of the compound or pharmaceutically acceptable salt thereof prior to and during administration of the compound or a pharmaceutically acceptable salt thereof to the subject.

In an aspect, provided herein is a method of treating the blood of a subject in need thereof, the method comprising administering to the subject an effective amount of a compound represented by

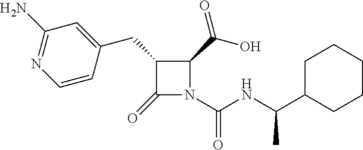 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In an aspect, provided herein is a method of maintaining the plasma level of a compound represented by

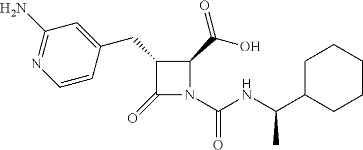 [SMILES: C[C@@H](NC(=O)N1C(=O)[C@H](Cc2ccnc(N)c2)[C@H]1C(=O)O)C1CCCCC1]

or a pharmaceutically acceptable salt thereof, in the blood of a subject in contact with an artificial surface, the method comprising:

(i) administering the compound or pharmaceutically acceptable salt thereof to the subject prior to or while contacting the artificial surface with the blood of the subject; and

(ii) conditioning an artificial surface with the compound or a pharmaceutically acceptable salt thereof prior to or while contacting the artificial surface with the blood of the subject;

thereby maintaining the plasma level of the compound or a pharmaceutically acceptable salt thereof in the blood of the subject.

In some embodiments of the methods described herein, the compound, or a pharmaceutically acceptable salt thereof, maintains a constant activated partial thromboplastin time (aPTT) in the blood of the subject before and after contact with the artificial surface. In some embodiments, the compound or a pharmaceutically acceptable salt thereof is administered to the subject prior to and while contacting the artificial surface with the blood of the subject.

In some embodiments, the artificial surface is conditioned with the compound or a pharmaceutically acceptable salt thereof prior to and while contacting the artificial surface with the blood of the subject. In some embodiments, the method further prevents or reduces risk of a blood clot formation in the blood of the subject in contact with the artificial surface.

In some embodiments, the artificial surface is a cardiopulmonary bypass circuit. In some embodiments, the artificial surface is an extracorporeal membrane oxygenation (ECMO) apparatus. In some embodiments, the ECMO apparatus is venovenous ECMO apparatus or venoarterial ECMO apparatus.

In an aspect, provided herein is a method of preventing or reducing a risk of a thromboembolic disorder in a subject during or after a medical procedure, comprising:

(i) administering to the subject an effective amount of a compound represented by:

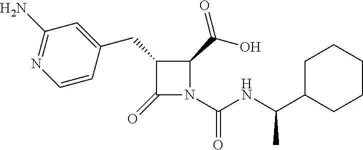 [SMILES: C[C@@H](NC(=O)N1C(=O)[C@H](Cc2ccnc(N)c2)[C@H]1C(=O)O)C1CCCCC1]

or pharmaceutically acceptable salt thereof, before, during, or after the medical procedure; and

(ii) contacting blood of the subject with an artificial surface;

thereby preventing or reducing the risk of the thromboembolic disorder during or after the medical procedure.

In some embodiments, the artificial surface is conditioned with the compound or pharmaceutically acceptable salt thereof prior to administration of the compound to the subject prior to, during, or after the medical procedure.

In some embodiments, the artificial surface is conditioned with a solution comprising the compound or a pharmaceutically acceptable salt thereof prior to administration of the compound or a pharmaceutically acceptable salt thereof to the subject prior to, during, or after the medical procedure. In some embodiments, the solution is a saline solution, Ringer's solution, or blood. In some embodiments, the solution further comprises blood. In some embodiments, the blood is acquired from the subject or a donor.

In some embodiments, the thromboembolic disorder is a blood clot.

In some embodiments, the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

In some embodiments, the medical procedure comprises a cardiopulmonary bypass. In some embodiments, the medical procedure comprises an oxygenation and pumping of blood via extracorporeal membrane oxygenation (ECMO). In some embodiments, the ECMO is venovenous ECMO or venoarterial ECMO.

In one aspect, the present invention is directed to a method of reducing the risk of stroke (e.g., ischemia, e.g., a transient ischemic event, large vessel acute ischemic stroke) in a subject that has suffered an ischemic event (e.g., a transient ischemic event), comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the administering reduces the risk of stroke (e.g., large vessel acute ischemic stroke) in a subject as compared to a subject who is not administered with the compound. In some embodiments, the administering reduces the risk of atrial fibrillation in a subject as compared to a subject who is not administered with the compound.

In one aspect, the present invention is directed to a method of reducing non-central nervous system systemic embolism (e.g., ischemia, e.g., a transient ischemic event) in a subject that has suffered an ischemic event (e.g., a transient ischemic event), comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the administering reduces non-central nervous system systemic embolism in a subject as compared to a subject who is not administered with the compound.

In one aspect, the present invention is directed to a method of treating deep vein thrombosis comprising administering to the subject that has suffered an ischemic event (e.g., a transient ischemic event), an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of prophylaxis of deep vein thrombosis comprising administering to the subject that has suffered a deep vein thrombosis (e.g., a subject that has been previously treated for a deep vein thrombosis), an effective amount of Compound 1 or a pharmaceutically acceptable salt thereof, or of a composition described herein (e.g., a composition comprising Compound 1 or a pharmaceutically acceptable salt thereof). In one aspect, the present invention is directed to a method of reducing the risk of recurrence of deep vein thrombosis comprising administering to the subject that has suffered a deep vein thrombosis (e.g., a subject that has been previously treated for a deep vein thrombosis), an effective amount of Compound 1 or a pharmaceutically acceptable salt thereof, or of a composition described herein (e.g., a composition comprising Compound 1 or a pharmaceutically acceptable salt thereof). In some embodiments, the administering reduces the risk of recurrence of deep vein thrombosis in a subject as compared to a subject who is not administered with the compound.

In one aspect, the present invention is directed to a method of prophylaxis of venous thromboembolism, e.g., deep vein thrombosis or pulmonary embolism in a subject, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the subject is undergoing surgery. In some embodiments, the subject is administered the compound, pharmaceutically acceptable salt thereof, or composition thereof before, during, or after surgery. In some embodiments, the subject is undergoing knee or hip replacement surgery. In some embodiments, the subject is undergoing orthopedic surgery. In some embodiments, the subject is undergoing lung surgery. In some embodiments, the subject is being treated for cancer, e.g., by surgery. In some embodiments, the subject is suffering from a chronic medical condition. In some embodiments, the venous thromboembolism is associated with cancer. In some embodiments, the compound, pharmaceutically acceptable salt thereof, or composition described herein is a primary agent in prophylaxis of the deep vein thrombosis or venous thromboembolism. In some embodiments, the compound, pharmaceutically acceptable salt thereof, or composition described herein is used as an extended therapy. In one aspect, the present invention is directed to a method of reducing the risk of venous thromboembolism, e.g., deep vein thrombosis or pulmonary embolism, in a subject, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the subject is undergoing surgery. In some embodiments, the subject is administered the compound, pharmaceutically acceptable salt thereof, or composition thereof after surgery. In some embodiments, the subject is undergoing knee or hip replacement surgery. In some embodiments, the subject is undergoing orthopedic surgery. In some embodiments, the subject is undergoing lung surgery. In some embodiments, the subject is being treated for cancer, e.g., by surgery. In some embodiments, the subject is suffering from a chronic medical condition. In some embodiments, the thromboembolic disorder is associated with cancer. In some embodiments, the compound, pharmaceutically acceptable salt thereof, or composition described herein is a primary agent in reducing the risk of the thromboembolic disorder. In some embodiments, the compound, pharmaceutically acceptable salt thereof, or composition described herein is used as an extended therapy.

In one aspect, the present invention is directed to a method of reducing the risk of stroke (e.g., large vessel acute ischemic stroke) or systemic embolism in a subject in need thereof, comprising administering to the subject an effective amount of a compound described herein, e.g, Compound 1, or a pharmaceutically acceptable salt thereof, or a composition described herein, e.g., a composition comprising Compound 1. In some embodiments, the subject is suffering from atrial fibrillation (e.g., non-valvular atrial fibrillation). In some embodiments, the subject is suffering from a renal disorder (e.g., end-stage renal disease).

In one aspect, the present invention is directed to a method of prophylaxis of stroke (e.g., large vessel acute ischemic stroke) or systemic embolism in a subject in need thereof, comprising administering to the subject an effective amount of a compound described herein, e.g, Compound 1, or a pharmaceutically acceptable salt thereof, or a composition described herein, e.g., a composition comprising Compound 1. In some embodiments, the subject is suffering from atrial fibrillation (e.g., non-valvular atrial fibrillation). In some embodiments, the subject is suffering from a renal disorder (e.g., end-stage renal disease).

In one aspect, the present invention is directed to a method of reducing the risk of recurrence of pulmonary embolism (e.g., symptomatic pulmonary embolism) comprising administering to the subject that has suffered a pulmonary embolism (e.g., a subject that has been previously treated for a pulmonary embolism), an effective amount of Compound 1 or a pharmaceutically acceptable salt thereof, or of a composition described herein (e.g., a composition comprising Compound 1 or a pharmaceutically acceptable salt thereof). In some embodiments, the administering reduces the risk of recurrence of pulmonary embolism in a subject as compared to a subject who is not administered with the compound.

In one aspect, the present invention is directed to a method of prophylaxis of pulmonary embolism in a subject that has suffered a pulmonary embolism (e.g., a subject that has been previously treated for a pulmonary embolism), comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of reducing the risk of recurrence of pulmonary embolism (e.g., symptomatic pulmonary embolism) comprising administering to the subject that has suffered a deep vein thrombosis (e.g., a subject that has been previously treated for a deep vein thrombosis), an effective amount of Compound 1 or a pharmaceutically acceptable salt thereof, or of a composition described herein (e.g., a composition comprising Compound 1 or a pharmaceutically acceptable salt thereof). In some embodiments, the administering reduces the risk of recurrence of pulmonary embolism in a subject as compared to a subject who is not administered with the compound.

In one aspect, the present invention is directed to a method of prophylaxis of pulmonary embolism in a subject that has suffered a deep vein thrombosis (e.g., a subject that has been previously treated for a deep vein thrombosis), comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention features a method of treating deep vein thrombosis in a subject that has been previously administered an anticoagulant, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the anticoagulant was administered parenterally for 5-10 days.

In one aspect, the present invention features a method of treating a pulmonary embolism in a subject that has been previously administered an anticoagulant, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the anticoagulant was administered parenterally for 5-10 days.

In one aspect, the present invention is directed to a method of treating a subject that has had an ischemic event (e.g., transient ischemia), comprising: administering Compound 1 or a pharmaceutically acceptable salt thereof, or of a composition described herein (e.g., a composition comprising Compound 1 or a pharmaceutically acceptable salt thereof) to the subject. In some embodiments, the compound is administered to the subject within 24 hours or less, e.g., 12, 10, 9, 8, 7, 6 hours or less, after the onset of the ischemic event in the subject.

In one aspect, the present invention is directed to a method of treating a subject that has had an ischemic event (e.g., transient ischemia), comprising: administering Compound 1 or a pharmaceutically acceptable salt thereof, or of a composition described herein (e.g., a composition comprising Compound 1 or a pharmaceutically acceptable salt thereof) to the subject. In some embodiments, the compound is administered to the subject within more than 2 hours to 12 hours, e.g., more than 2 hours to 10 hours or less, more than 2 hours to 8 hours or less, after the onset of the ischemic event in the subject.

In one aspect, the present invention is directed to a method of treating hypertension, e.g., arterial hypertension, in a subject, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the hypertension, e.g., arterial hypertension, results in atherosclerosis. In some embodiments, the hypertension is pulmonary arterial hypertension.

In one aspect, the present invention is directed to a method of reducing the risk of hypertension, e.g., arterial hypertension, in a subject, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the hypertension, e.g., arterial hypertension, results in atherosclerosis. In some embodiments, the hypertension is pulmonary arterial hypertension.

In one aspect, the present invention is directed to a method of prophylaxis of hypertension, e.g., arterial hypertension, in a subject, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the hypertension, e.g., arterial hypertension, results in atherosclerosis. In some embodiments, the hypertension is pulmonary arterial hypertension.

In one aspect, the present invention is directed to a method of reducing inflammation in a subject, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the inflammation is vascular inflammation. In some embodiments, the vascular inflammation is accompanied by atherosclerosis. In some embodiments, the vascular inflammation is accompanied by a thromboembolic disease in the subject. In some embodiments, the vascular inflammation is angiotensin II-induced vascular inflammation.

In one aspect, the present invention is directed to a method of preventing vascular leukocyte infiltration in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of preventing angiotensin II-induced endothelial dysfunction in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of preventing thrombin propagation in a subject, comprising administering to the subject an effective amount of Compound 1 or a pharmaceutically acceptable salt thereof, or of a composition described herein (e.g., a composition comprising Compound 1 or a pharmaceutically acceptable salt thereof). In some embodiments, the thrombin propagation occurs on platelets.

In one aspect, the present invention is directed to a method of treating hypertension-associated renal dysfunction in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of prophylaxis of hypertension-associated renal dysfunction in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of reducing the risk of hypertension-associated renal dysfunction in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of treating kidney fibrosis in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of prophylaxis of kidney fibrosis in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of reducing the risk of kidney fibrosis in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of treating kidney injury in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of prophylaxis of kidney injury in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of reducing the risk of kidney injury in a subject, comprising administering to the subject an effective amount of Compound 1 or a pharmaceutically acceptable salt thereof, or of a composition described herein (e.g., a composition comprising Compound 1 or a pharmaceutically acceptable salt thereof). In one aspect, the present invention is directed to a method of inhibiting Factor XIa in a subject, comprising administering to the subject that has suffered ischemia an effective amount of Compound 1 or a pharmaceutically acceptable salt thereof, or of a composition described herein (e.g., a composition comprising Compound 1 or a pharmaceutically acceptable salt thereof). In some embodiments, the ischemia is coronary ischemia. In some embodiments, the subject is a mammal (e.g., a human) In some embodiments, the subject is undergoing surgery (e.g., knee replacement surgery or hip replacement surgery). In some embodiments, the ischemia is coronary ischemia. In some embodiments, the subject is a subject with non-valvular atrial fibrillation. In some embodiments, the subject has one or more of the following risk factors for stroke: a prior stroke (e.g., ischemic, unknown, hemorrhagic), transient ischemic attack, or non-CNS systemic embolism. In some embodiments, the subject has one or more of the following risk factors for stroke: 75 years or older of age, hypertension, heart failure or left ventricular ejection fraction (e.g., less than or equal to 35%), or diabetes mellitus.

In some embodiments, the compound is administered by oral or parenteral (e.g., intravenous) administration. In some embodiments, the compound is administered by oral administration. In some embodiments, the compound is administered by parenteral (e.g., intravenous) administration. In some embodiments, the compound is administered by subcutaneous administration.

In some embodiments, the compound is administered prior to an ischemic event (e.g., to a subject is at risk of an ischemic event).

In some embodiments, the compound is administered after an ischemic event (e.g., a transient ischemic event). In some embodiments, the compound is administered about 1, 2, 3, 4, 5, 6, 7, 8, 9, 10, 11, 12, 13, or 14 days or more after an ischemic event (e.g., a transient ischemic event). In some embodiments, the compound is administered about 1, 2, 3, 4, 5, 6, 7, or 8 weeks or more after an ischemic event (e.g., a transient ischemic event).

In some embodiments, the compound is administered in combination with an additional therapeutic agent. In some embodiments, the additional therapeutic agent is administered after administration of the compound. In some embodiments, the additional therapeutic agent is administered orally. In some embodiments, the additional therapeutic agent is administered at least 1, 2, 3, 4, 5, 6, 7, 8, 10, 12, 14, 16, 18, 20, or 24 hours or more after administration of the compound. In some embodiments, the additional therapeutic agent is administered at least 1, 2, 3, 4, 5, 6, 7, 14, 21, or 28 days or more after administration of the compound. In some embodiments, the additional therapeutic agent is administered about 1 day, about 2 days, about 3 days, about 4 days, about 5 days, about 6 days, about 7 days or more after administration of the compound.

In some embodiments, the additional therapeutic agent is administered chronically (e.g., for about 1 day, about 2 days, about 3 days, about 4 days, about 5 days, about 6 days, about 7 days, about 8 days, about 9 days, about 10 days, about 11 days, about 12 days, about 13 days, or about 14 days or more) after administration of the compound.

In some embodiments, the additional therapeutic agent treats a side effect (e.g., active pathological bleeding or severe hypersensitivity reactions (e.g., anaphylactic reactions), spinal and or epidural hematoma, gastrointestinal disorder (e.g., abdominal pain upper, dyspepsia, toothache), general disorders and administration site conditions (e.g., fatigue), infections and infestations (e.g., sinusitis, urinary tract infection), musculoskeletal and connective tissues disorders (e.g., back pain, osteoarthritis), respiratory, thoracic and mediastinal disorders (e.g., oropharyngeal pain), injury, poisoning, and procedural complications (e.g., wound secretion), musculoskeletal and connective tissues disorders (e.g., pain in extremity, muscle spasm), nervous system disorders (e.g., syncope), skin and subcutaneous tissue disorders (e.g., pruritus, blister), blood and lymphatic system disorders (e.g., agranulocytosis), gastrointestinal disorders (e.g., retroperitoneal hemorrhage), hepatobiliary disorders (e.g., jaundice, cholestasis, cytolytic hepatitis), immune system disorders (e.g., hypersensitivity, anaphylactic reaction, anaphylactic shock, angioedema), nervous system disorders (e.g., cerebral hemorrhage, subdural hematoma, epidural hematoma, hemiparesis), skin and subcutaneous tissue disorders (e.g., Stevens-Johnson syndrome).

In some embodiments, the additional therapeutic agent is a NSAID (e.g., aspirin or naproxen), platelet aggregation inhibitor (e.g., clopidogrel), or anticoagulant (e.g., warfarin or enoxaparin).

In some embodiments, the additional therapeutic agent results in an additive therapeutic effect. In some embodiments, the additional therapeutic agent results in a synergistic therapeutic effect.

In another aspect, the present invention features a pharmaceutical composition comprising a compound described herein (e.g., Compound 1) and a pharmaceutically acceptable excipient.

In another aspect, the present invention features a method of modulating (e.g., inhibiting) Factor XIa in a patient. The method comprises the step of administering an effective amount of a compound described herein (e.g., Compound 1) or a pharmaceutically acceptable salt thereof, or of a composition described herein (e.g., a composition comprising Compound 1 or a pharmaceutically acceptable salt thereof)to a patient in need thereof, thereby modulating (e.g., inhibiting) Factor XIa.

In another aspect, the present invention features a method of treating a subject in need thereof for a thromboembolic disorder. The method comprises administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In another aspect, the present invention features a method of prophylaxis of a thromboembolic disorder in a subject. The method comprises administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In another aspect, the present invention features a method of reducing the risk of a thromboembolic disorder in a subject. The method comprises administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In one aspect, the present invention is directed to a method of treating end-stage renal disease in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of prophylaxis of end-stage renal disease in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In one aspect, the present invention is directed to a method of reducing the risk of end-stage renal disease in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In another aspect, the present invention features a method of treating a thromboembolic disorder in a subject need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of reducing the risk of a thromboembolic disorder in a subject need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of prophylaxis of a thromboembolic disorder in a subject need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of treating atrial fibrillation, in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). In some embodiments, the subject is also in need of dialysis, e.g., renal dialysis. In some embodiments, the compound described herein is administered to the subject while the subject is undergoing dialysis. In some embodiments, the compound or pharmaceutically acceptable salt or composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of reducing the risk of atrial fibrillation, in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). In some embodiments, the subject is at a high risk of developing atrial fibrillation. In some embodiments, the subject is also in need of dialysis, e.g., renal dialysis. In some embodiments, the compound described herein is administered to the subject while the subject is undergoing dialysis. In some embodiments, the compound or pharmaceutically acceptable salt or composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of prophylaxis of atrial fibrillation, in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). In some embodiments, the subject is at a high risk of developing atrial fibrillation. In some embodiments, the subject is also in need of dialysis, e.g., renal dialysis. In some embodiments, the compound described herein is administered to the subject while the subject is undergoing dialysis. In some embodiments, the compound or pharmaceutically acceptable salt or composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of treating heparin-induced thrombocytopenia in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof).

In another aspect, the present invention features a method of reducing the risk of heparin-induced thrombocytopenia in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof).

In another aspect, the present invention features a method of prophylaxis of heparin-induced thrombocytopenia in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof).

In another aspect, the present invention features a method of treating heparin-induced thrombocytopenia thrombosis in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof).

In another aspect, the present invention features a method of reducing the risk of heparin-induced thrombocytopenia thrombosis in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof).

In another aspect, the present invention features a method of prophylaxis of heparin-induced thrombocytopenia thrombosis in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof).

In another aspect, the present invention features a method of prophylaxis of a thromboembolic disorder in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof), wherein the subject has cancer or is being with a chemotherapeutic. In some embodiments, the subject is concurrently receiving chemotherapy. In some embodiments, the subject has elevated lactase dehydrogenase levels. In some embodiments, the thromboembolic disorder is venous thromboembolism. In some embodiments, the thromboembolic disorder is deep vein thrombosis. In some embodiments, the thromboembolic disorder is pulmonary embolism.

In another aspect, the present invention features a method of treating thrombotic microangiopathy in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of reducing the risk of thrombotic microangiopathy in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of prophylaxis of thrombotic microangiopathy in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of prophylaxis of recurrent ischemia in a subject in need thereof, the method comprising administering to the subject an effective amount of a compound described herein (e.g., Compound 1) or a pharmaceutically acceptable salt thereof, or of a composition described herein (e.g., a composition comprising Compound 1 or a pharmaceutically acceptable salt thereof), wherein the subject has acute coronary syndrome. In some embodiments, the subject has atrial fibrillation. In some embodiments, the subject does not have atrial fibrillation. In another aspect, the present invention features a method of treating a subject identified as being at risk, e.g., high risk, for stroke (e.g., large vessel acute ischemic stroke) or thrombosis thereby reducing the likelihood of stroke (e.g., large vessel acute ischemic stroke) or thrombosis in the subject. In some embodiments, the subject is further identified as being at risk for bleeding (e.g., excessive bleeding) or sepsis. In some embodiments, the treatment is effective without bleeding liabilities. In some embodiments, the treatment is effective to maintain the patency of infusion ports and lines. In addition, the compounds described herein (e.g., Compound 1) are useful in the treatment and prevention of other diseases in which the generation of thrombin has been implicated as playing a physiologic role. For example, thrombin has been implicated in contributing to the morbidity and mortality of chronic and degenerative diseases, such as cancer, arthritis, atherosclerosis, vascular dementia, and Alzheimer's disease, by its ability to regulate many different cell types through specific cleavage and activation of a cell surface thrombin receptor, mitogenic effects, diverse cellular functions such as cell proliferation, for example, abnormal proliferation of vascular cells resulting in restenosis or angiogenesis, release of PDGF, and DNA synthesis. Inhibition of Factor XIa effectively blocks thrombin generation and therefore neutralizes any physiologic effects of thrombin on various cell types. The representative indications discussed above include some, but not all, of the potential clinical situations amenable to treatment with a Factor XIa inhibitor.

In another aspect, the present invention features a method of treating a subject that has edema (e.g., angioedema, e.g., hereditary angioedema), comprising administering Compound 1 or a pharmaceutically acceptable salt thereof, or a composition described herein (e.g., a composition comprising Compound 1 or a pharmaceutically acceptable salt thereof) to the subject.

In another aspect, the present invention features a method of prophylaxis of edema (e.g., angioedema, e.g., hereditary angioedema) in a subject, comprising administering Compound 1 or a pharmaceutically acceptable salt thereof, or a composition described herein (e.g., a composition comprising Compound 1 or a pharmaceutically acceptable salt thereof) to the subject.

In another aspect, the present invention features a method of reducing the risk of edema (e.g., angioedema, e.g., hereditary angioedema) in a subject, comprising administering Compound 1 or a pharmaceutically acceptable salt thereof, or a composition described herein (e.g., a composition comprising Compound 1 or a pharmaceutically acceptable salt thereof) to the subject.

In another aspect, the present invention features a method of inhibiting kallikrein in a subject, comprising administering to the subject with edema (e.g., angioedema, e.g., hereditary angioedema), an effective amount of Compound 1 or a pharmaceutically acceptable salt thereof, or of a composition described herein (e.g., a composition comprising Compound 1 or a pharmaceutically acceptable salt thereof) to the subject.

In another aspect, the present invention features a method of treating a thromboembolic consequence or complication in a subject, comprising administering to a subject an effective amount of Compound 1 or a pharmaceutically acceptable salt thereof, or of a composition described herein (e.g., a composition comprising Compound 1 or a pharmaceutically acceptable salt thereof). In some embodiments, the thromboembolic consequence or complication is associated with a peripheral vascular intervention (e.g., of the limbs), hemodialysis, catheter ablation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the present invention features a method of prophylaxis of a thromboembolic consequence or complication in a subject, comprising administering to a subject an effective amount of Compound 1 or a pharmaceutically acceptable salt thereof, or of a composition described herein (e.g., a composition comprising Compound 1 or a pharmaceutically acceptable salt thereof). In some embodiments, the thromboembolic consequence or complication is associated with a peripheral vascular intervention (e.g., of the limbs), hemodialysis, catheter ablation, e.g., catheter ablation for atrial fibrillation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the present invention features a method of reducing the risk of a thromboembolic consequence or complication in a subject, comprising administering to a subject an effective amount of Compound 1 or a pharmaceutically acceptable salt thereof, or of a composition described herein (e.g., a composition comprising Compound 1 or a pharmaceutically acceptable salt thereof). In some embodiments, the thromboembolic consequence or complication is associated with a peripheral vascular intervention (e.g., of the limbs), hemodialysis, catheter ablation, e.g., catheter ablation for atrial fibrillation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the invention features a method of treating restenosis following arterial injury in a subject, comprising administering to a subject an effective amount of Compound 1 or a pharmaceutically acceptable salt thereof, or of a composition described herein (e.g., a composition comprising Compound 1 or a pharmaceutically acceptable salt thereof). In some embodiments, the arterial injury occurs after a cranial artery stenting.

In another aspect, the present invention features a method of prophylaxis of restenosis following arterial injury in a subject, comprising administering to a subject an effective amount of Compound 1 or a pharmaceutically acceptable salt thereof, or of a composition described herein (e.g., a composition comprising Compound 1 or a pharmaceutically acceptable salt thereof). In some embodiments, the arterial injury occurs after a cranial artery stenting.

In another aspect, the present invention features a method of reducing the risk of restenosis following arterial injury in a subject, comprising administering to a subject an effective amount of Compound 1 or a pharmaceutically acceptable salt thereof, or of a composition described herein (e.g., a composition comprising Compound 1 or a pharmaceutically acceptable salt thereof). In some embodiments, the arterial injury occurs after a cranial artery stenting.

In another aspect, the present invention features a method of treating hepatic vessel thrombosis in a subject, comprising administering to a subject an effective amount of Compound 1 or a pharmaceutically acceptable salt thereof, or of a composition described herein (e.g., a composition comprising Compound 1 or a pharmaceutically acceptable salt thereof).

In another aspect, the present invention features a method of prophylaxis of hepatic vessel thrombosis in a subject, comprising administering to a subject an effective amount of Compound 1 or a pharmaceutically acceptable salt thereof, or of a composition described herein (e.g., a composition comprising Compound 1 or a pharmaceutically acceptable salt thereof).

In another aspect, the present invention features a method of reducing the risk of hepatic vessel thrombosis in a subject, comprising administering to a subject an effective amount of Compound 1 or a pharmaceutically acceptable salt thereof, or of a composition described herein (e.g., a composition comprising Compound 1 or a pharmaceutically acceptable salt thereof).

In another aspect, the present invention features a method of treating a non-ST-elevation myocardial infarction or ST-elevation myocardial infarction), comprising administering to a subject an effective amount of Compound 1 or a pharmaceutically acceptable salt thereof, or of a composition described herein (e.g., a composition comprising Compound 1 or a pharmaceutically acceptable salt thereof).

In another aspect, the present invention features a method of prophylaxis of a non-ST-elevation myocardial infarction or ST-elevation myocardial infarction in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In another aspect, the present invention features a method of reducing the risk of a non-ST-elevation myocardial infarction or ST-elevation myocardial infarction in a subject, comprising administering to the subject an effective amount of Compound 1 or a pharmaceutically acceptable salt thereof, or of a composition described herein (e.g., a composition comprising Compound 1 or a pharmaceutically acceptable salt thereof).

In another aspect, the present invention features a method of maintaining blood vessel patency, comprising administering to a subject an effective amount of Compound 1 or a pharmaceutically acceptable salt thereof, or of a composition described herein (e.g., a composition comprising Compound 1 or a pharmaceutically acceptable salt thereof). In some embodiments, the subject has acute kidney injury. In some embodiments, the subject additionally undergoes continuous renal replacement therapy.

In some embodiments of any of the foregoing, the compound described herein or composition thereof is administered orally or parenterally. In certain embodiments, the compound or composition thereof is administered orally. In certain embodiments, the compound or composition thereof is administered after the subject has discontinued use of a direct oral anticoagulant. In certain embodiments, the subject used the direct oral anticoagulant for up to about 2.5 years. In certain embodiments, the subject is a mammal, e.g., a human.

In some embodiments of the methods described herein, the pharmaceutically acceptable salt of the compound is a hydrochloride salt. In some embodiments, the compound is administered to the subject intravenously. In some embodiments, the compound is administered to the subject subcutaneously. In some embodiments, the compound is administered to the subject as a continuous intravenous infusion. In some embodiments, the compound is administered to the subject as a bolus. In some embodiments, the subject is a human. In some embodiments, the subject has an elevated risk of a thromboembolic disorder. In some embodiments, the thromboembolic disorder is a result of a complication in surgery.

In some embodiments, the subject is sensitive to or has developed sensitivity to heparin. In some embodiments, the subject is resistant to or has developed resistance to heparin.

In some embodiments, the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

### BRIEF DESCRIPTION OF THE DRAWINGS

FIG. 1 depicts an exemplary 1H NMR spectrum of Sample #4 obtained from the procedure in Example 1.

FIG. 2 depicts an exemplary 1H NMR spectrum of Sample #8 obtained from the procedure in Example 3, Step 1.

FIG. 3 depicts an exemplary 1H NMR spectrum of crude product obtained from the procedure in Example 3, Step 2.

FIG. 4 depicts an exemplary 1H NMR spectrum of purified product obtained from the procedure in Example 3, Step 2.

FIG. 5 depicts an exemplary 1H NMR spectrum of the product obtained from the procedure in Example 3, Step 3.

FIG. 6 depicts an exemplary 1H NMR spectrum of the product obtained from the procedure in Example 3, Step 4.

FIG. 7 depicts an exemplary 13C NMR spectrum of the product obtained from the procedure in Example 3, Step 4.

FIG. 8 depicts an expansion of the 13C NMR spectrum in the 105-180 ppm region of FIG. 7.

FIG. 9 depicts an exemplary XRPD pattern for the product obtained from the procedure in Example 3, Step 4.

FIG. 10 depicts the pressure gradient across membrane oxygenator for cardiopulmonary bypass experiment conducted in the hound model.

FIG. 11 depicts a comparison of plasma concentrations and activated partial thromboplastin time (aPTT) ratio measured in the hound model.

FIG. 12 depicts the activated partial thromboplastin time (aPTT) measured in the hound model following Compound 1 administration.

### DETAILED DESCRIPTION

### Definitions

The term “agitated” as used herein refers to any motion of a macroscopic constituent of the reaction mixture which is induced from outside, relative to another macroscopic constituent of the reaction mixture. The term “stirring” as used herein refers to any motion of a macroscopic constituent of the reaction mixture which is induced from outside via a stirring device, relative to another macroscopic constituent of the reaction mixture, e.g., induction stirring, and can include normal, internal stirring procedures known to one of skill in the art. As used herein, “XRPD” refers to X-ray powder diffraction.

As used herein, “slurrying” refers to a method wherein a compound as described herein is suspended in a solvent (e.g., polar aprotic solvent or nonpolar solvent) and is collected again (e.g., by filtration) after agitating the suspension.

As used herein, “crystalline” refers to a solid having a highly regular chemical structure. The molecules are arranged in a regular, periodic manner in the 3-dimensional space of the lattice.

The term “substantially crystalline” refers to forms that may be at least a particular weight percent crystalline. Particular weight percentages are 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, Compound 1 can be a substantially crystalline sample of any of the crystalline solid forms described herein.

The term “substantially pure” relates to the composition of a specific crystalline solid form of Compound 1 that may be at least a particular weight percent free of impurities and/or other solid forms of Compound 1 or a pharmaceutically acceptable salt thereof. Particular weight percentages are 70%, 75%, 80%, 85%, 90%, 95%, 99%, or any percentage between 70% and 100%. In some embodiments, a crystalline solid form of Compound 1 or a pharmaceutically acceptable salt thereof as described herein is substantially pure at a weight percent between 95% and 100%, e.g., about 95%, about 96%, about 97%, about 98%, about 99%, or about 99.9%.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also, “therapeutic treatment”).

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

Disease, disorder, and condition are used interchangeably herein.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. In some embodiments, the pediatric subject is between the age of 0 and 18 years old. In some embodiments, the adult subject is beyond 18 years old.

As used herein, the term “artificial surface” refers to any non-human or non-animal surface that comes into contact with blood of the subject, for example, during a medical procedure. It can be a vessel for collecting or circulating blood of a subject outside the subject's body. It can also be a stent, valve, intraluminal catheter or a system for pumping blood. By way of non-limiting example such artificial surfaces can be steel, any type of plastic, glass, silicone, rubber, etc. In some embodiments, the artificial surface is exposed to at least 50%. 60%, 70% 80%, 90% or 100% of the blood of subject.

As used herein, the term “conditioning” or “conditioned” with respect to an artificial surface refers to priming or flushing the artificial surface (e.g., extracorporeal surface) with a compound described herein (e.g., Compound 1) or a pharmaceutically acceptable salt thereof, already in a priming or flushing solution (e.g., blood, a saline solution, Ringer's solution) or as a separate administration to the artificial surface prior to, during, or after a medical procedure.

### Compounds

Described herein are compounds that inhibit Factor XIa or kallikrein.

In one aspect, the present invention is directed to Compound 1:

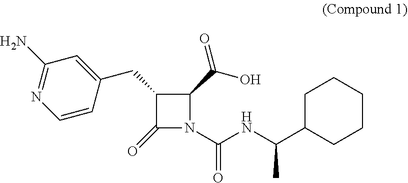 [SMILES: C[C@@H](NC(=O)N1C(=O)[C@H](Cc2ccnc(N)c2)[C@H]1C(=O)O)C1CCCCC1]

or a pharmaceutically acceptable salt thereof, e.g., a hydrochloride salt of Compound 1. In some embodiments, Compound 1 or a pharmaceutically acceptable salt thereof is crystalline.

In some embodiments, Compound 1 or a pharmaceutically acceptable salt thereof exists as a substantially pure crystalline solid form.

In one aspect, provided herein is a pharmaceutically acceptable salt of Formula (I):

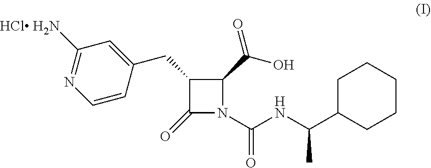 [SMILES: C[C@@H](NC(=O)N1C(=O)[C@H](Cc2ccnc(N)c2)[C@H]1C(=O)O)C1CCCCC1]

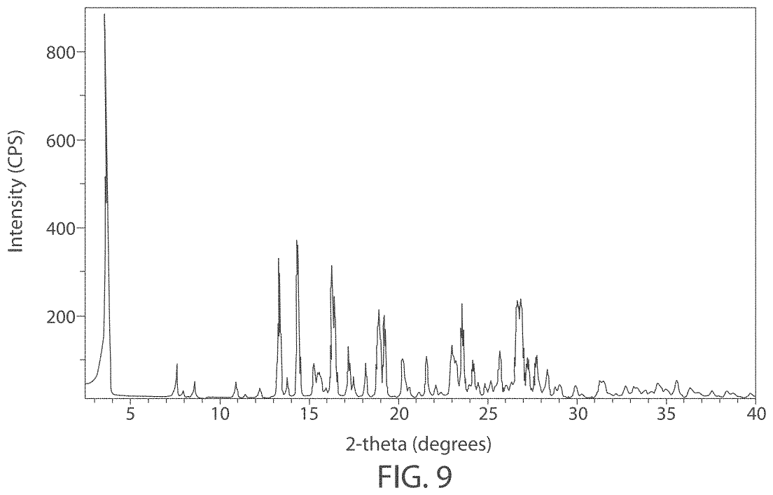

The pharmaceutically acceptable salt of Formula (I) is a hydrochloride salt of Compound 1 and also referred to herein as Compound 1.HCl. In some embodiments, Compound 1.HCl is crystalline. In some embodiments, Compound 1.HCl exists as a substantially pure crystalline solid form. In some embodiments, Compound 1.HCl has an XRPD pattern substantially as depicted in FIG. 9.

In one aspect, provided herein is a crystalline pharmaceutically acceptable salt of the Formula (I):

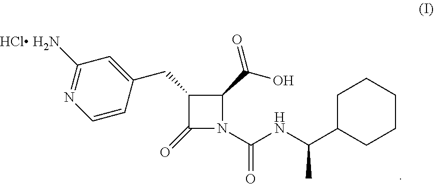 [SMILES: C[C@@H](NC(=O)N1C(=O)[C@H](Cc2ccnc(N)c2)[C@H]1C(=O)O)C1CCCCC1]

In some embodiments, a compound described herein is formed into a salt. A compound described herein can be administered as a free acid, a zwitterion or as a salt. A salt can also be formed between a cation and a negatively charged substituent on a compound described herein, the deprotonated carboxylic acid moiety of Compound 1 for example. Suitable cationic counterions include sodium ions, potassium ions, magnesium ions, calcium ion, and ammonium ions (e.g., a tetraalkyl ammonium cation such as tetramethylammonium ion). In acid addition salts, a salt can be formed between an anion and a positively charged substituent (e g , amino group) or basic substituent (e.g., pyridyl) on a compound described herein. Suitable anions include chloride, bromide, iodide, sulfate, nitrate, phosphate, citrate, methanesulfonate, trifluoroacetate, and acetate.

Pharmaceutically acceptable salts of the compounds described herein (e.g., a pharmaceutically acceptable salt of Compound 1) also include those derived from pharmaceutically acceptable inorganic and organic acids and bases. Examples of suitable acid salts include acetate, 4-acetamidobenzoate, adipate, alginate, 4-aminosalicylate, aspartate, ascorbate, benzoate, benzenesulfonate, bisulfate, butyrate, citrate, camphorate, camphorsulfonate, carbonate, cinnamate, cyclamate, decanoate, decanedioate, 2,2-dichloroacetate, digluconate, dodecylsulfate, ethanesulfonate, ethane-1,2-disulfonate, formate, fumarate, galactarate, glucoheptanoate, gluconate, glucoheptonate, glucoronate, glutamate, glutarate, glycerophosphate, glycolate, hemisulfate, heptanoate, hexanoate, hippurate, hydrochloride, hydrobromide, hydroiodide, 1-hydroxy-2-naphthoate, 2-hydroxyethanesulfonate, isobutyrate, lactate, lactobionate, laurate, malate, maleate, malonate, mandelate, methanesulfonate, naphthalene-1,5-disulfonate, 2-naphthalenesulfonate, nicotinate, nitrate, octanoate, oleate, oxalate, 2-oxoglutarate, palmitate, palmoate, pectinate, 3-phenylpropionate, phosphate, phosphonate, picrate, pivalate, propionate, pyroglutamate, salicylate, sebacate, succinate, stearate, sulfate, tartrate, thiocyanate, toluenesulfonate, tosylate, and undecanoate.

Salts derived from appropriate bases include alkali metal (e.g., sodium), alkaline earth metal (e.g., magnesium), ammonium and (alkyl)4N+salts. This invention also envisions the quaternization of any basic nitrogen-containing groups of the compounds disclosed herein. Water or oil-soluble or dispersible products may be obtained by such quaternization.

As used herein, the compounds of this invention, including the Compound 1, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood), or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

Any formula or a compound described herein is also intended to represent unlabeled forms as well as isotopically labeled forms of the compounds, isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as 2H, 3H, 11C, 13C, 14C, 15N, 18F 51P, 32P, 35S, 36Cl, 125I respectively. The invention includes various isotopically labeled compounds as defined herein, for example, those into which radioactive isotopes, such as 3H, 13C, and 14C are present. Such isotopically labelled compounds are useful in metabolic studies (with 14C), reaction kinetic studies (with, for example ′H or 3H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies, isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of a formula described herein. The concentration of such a heavier isotope, specifically deuterium, may be defined by the isotopic enrichment factor. The term “isotopic enrichment factor” as used herein means the ratio between the isotopic abundance and the natural abundance of a specified isotope If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 8633.3 (99.5% deuterium incorporation).

Isotopically-labelled compounds described herein can generally be prepared by conventional techniques known to those skilled in the art or by processes analogous to those described in the accompanying Examples and Preparations using an appropriate isotopically-labeled reagents in place of the non-labeled reagent previously employed. Pharmaceutically acceptable solvates in accordance with the invention include those wherein the solvent of crystallization may be isotopically substituted, e.g, D2O, D6-acetone, D6-DMSO.

Any asymmetric atom (e.g., carbon or the like) of the compound(s) of the present invention can be present in racemic or enantiomerically enriched, for example the (R)- (S)- or (RS)-configuration, in certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form. Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof. Any resulting mixtures of isomers can be separated on the basis of the physicochemical differences of the constituents, into the pure or substantially pure geometric or optical isomers, diastereomers, racemates, for example, by chromatography or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. An acidic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, (+)-O,O′-Di-p-toluoyl-D-tartaric acid, mandelic acid, malic acid or camphor-10-sulfonic acid. Racemic products can also be resolved by chiral chromatography, e.g., high pressure liquid chromatography (HPLC) using a chiral adsorbent.

The compounds described herein (e.g., Compound 1) may also be represented in multiple tautomeric forms. In such instances, the invention expressly includes all tautomeric forms of the compounds described herein. All crystal forms of the compounds described herein are expressly included in this invention.

A compound described herein (e.g., Compound 1) can be evaluated for its ability to modulate (e.g., inhibit) Factor XIa or kallikrein.

### Good Manufacturing Practice

Good Manufacturing Practice (GMP) refers to all applicable standards relating to manufacture of pharmaceutical products as they apply to the manufacture of Supplied Material, and including (i) standards promulgated by any Regulatory Authority having jurisdiction over the Manufacture of the Supplied Material, in the form of Applicable Laws, including the U.S. current Good Manufacturing Practices regulations promulgated by the FDA, as described in 21 U.S.C. 351, 21 C.F.R. Parts 210 and 211, as amended, and any successor provision thereto and ICH Q7—Good Manufacturing Practice for Active Pharmaceutical Ingredients; (ii) standards promulgated by any Regulatory Authority having jurisdiction over the Manufacture of the Supplied Material, in the form of draft or final guidance documents (including advisory opinions, compliance policy guides and guidelines); and (iii) such other industry standards as may be agreed upon by the Parties in the Specifications (as defined and set forth in the Quality Agreement).

### Methods of Synthesizing Compounds

The compounds described herein can be synthesized by non-limiting conventional methods using commercially available starting materials and reagents. For example, compounds can be synthesized utilizing the methods set forth in U.S. Pat. No. 7,501,404, which is incorporated herein by reference, or using the methods described in the Examples herein.

Compounds described herein can be purified using various techniques in the art of synthetic organic chemistry. A compound described herein, e.g., a compound of any one of Formulae I, II, III, IV, V, VI, or VII, can be purified using one or more chromatographic methods, e.g., column chromatography or HPLC. A compound described herein, e.g., a compound of any one of Formulae I, II, III, IV, V, VI, or VII, can be purified by a purification method that is not chromatography, e.g., recrystallization or slurrying. In one embodiment, a compound described herein can be purified using recrystallization. In another embodiment, a compound described herein can also be purified by slurrying.

In some embodiments, a compound described herein that has been purified by chromatography can also be purified by a recrystallization. A compound described herein can also be purified by slurrying (or re-slurrying) the compound with one or more solvents, e.g., a slurry described herein. A compound described herein can also be purified by trituration with one or more solvents, e.g., a trituration described herein. For example, a compound described herein that has been purified by chromatography can also be purified by trituration. In a chemical reactor, the trituration process may be affected by suspension or resuspension of a solid product in a solvent or mixture of solvents with mechanical stirring. In an embodiment, a compound described herein can also be purified by precipitation from a solution using one or more anti-solvents. For example, a compound described herein that has been purified by chromatography can also be purified by precipitation. In one embodiment, a compound described herein is purified by simulated moving bed (SMB) chromatography. In one embodiment, a compound described herein is purified by supercritical fluid chromatography, e.g., supercritical fluid chromatography with liquid carbon dioxide. In one embodiment, a compound described herein is purified by chiral chromatography e.g., high pressure liquid chromatography (HPLC) using a chiral adsorbent.

In one aspect, provided herein is a process for preparing a pharmaceutically acceptable salt of Formula (I):

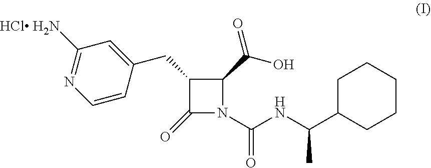 [SMILES: C[C@@H](NC(=O)N1C(=O)[C@H](Cc2ccnc(N)c2)[C@H]1C(=O)O)C1CCCCC1]

or a solvate (e.g., a hydrate) thereof, comprising dissolving a salt of Formula (II)

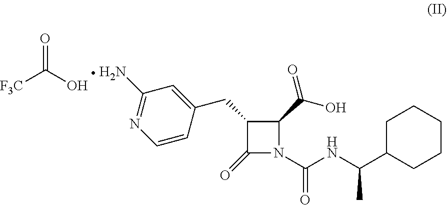 [SMILES: O=C(O)C(F)(F)F ; C[C@@H](NC(=O)N1C(=O)[C@H](Cc2ccnc(N)c2)[C@H]1C(=O)O)C1CCCCC1]

or a solvate (e.g., a hydrate) thereof in a solvent, thereby preparing a first solution, and adding hydrogen chloride to the first solution, thereby producing the pharmaceutically acceptable salt of Formula (I).

In some embodiments, the salt of Formula (II) is dissolved in an aprotic solvent. In some embodiments, the solvent comprises (e.g., consists of or consists essentially of) acetonitrile. In some embodiments, the hydrogen chloride is added to the first solution by bubbling HCl gas into the first solution or by adding a separate solution comprising HCl (e.g., an ethereal hydrochloric acid solution) to the first solution.

In some embodiments, the starting quantity of the salt of Formula (II) or solvate (e.g., a hydrate) thereof is greater than or equal to 500 grams. In some embodiments, the starting quantity of the salt of Formula (II) or solvate (e.g., a hydrate) thereof is greater than or equal to 1 kilogram. In some embodiments, the process produces over 300 grams (e.g., over about 350 grams (e.g., about 368 grams)) of pharmaceutically acceptable salt of Formula (I) or solvate (e.g., a hydrate) thereof.

In some embodiments, the process produces the pharmaceutically acceptable salt of Formula (I) or solvate (e.g., a hydrate) thereof in a yield greater than about 50% (e.g., in about 55% yield). In some embodiments, the process produces the pharmaceutically acceptable salt of Formula (I) or solvate (e.g., a hydrate) thereof in a yield greater than about 75%. In some embodiments, the process produces the pharmaceutically acceptable salt of Formula (I) or solvate (e.g., a hydrate) thereof in a yield greater than about 90%. In some embodiments, the process produces the pharmaceutically acceptable salt of Formula (I) or solvate (e.g., a hydrate) thereof in a yield greater than about 99%. In some embodiments, the purity of the pharmaceutically acceptable salt of Formula (I) or solvate (e.g., a hydrate) thereof is about 80%. In some embodiments, the purity of the pharmaceutically acceptable salt of Formula (I) or solvate (e.g., a hydrate) thereof is about 81%.

In some embodiments, the process further comprises purifying the pharmaceutically acceptable salt of Formula (I) or solvate (e.g., a hydrate) thereof by dissolving the pharmaceutically acceptable salt of Formula (I) or solvate (e.g., a hydrate) thereof in a solvent (e.g., isopropyl alcohol) followed by precipitation of the dissolved pharmaceutically acceptable salt of Formula (I) or solvate (e.g., a hydrate) thereof using another solvent (e.g., methyl tert-butyl ether). In some embodiments, the purity of the pharmaceutically acceptable salt of Formula (I) or solvate (e.g., a hydrate) thereof after precipitation is greater than 98%. In some embodiments, the purity of the pharmaceutically acceptable salt of Formula (I) or solvate (e.g., a hydrate) thereof after precipitation is about 98%.

In some embodiments, the process further comprises purifying the pharmaceutically acceptable salt of Formula (I) or solvate (e.g., a hydrate) thereof by slurrying the pharmaceutically acceptable salt of Formula (I) or solvate (e.g., a hydrate) thereof in a solvent (e.g., isopropyl alcohol) and then filtering the pharmaceutically acceptable salt of

Formula (I) or solvate (e.g., a hydrate) thereof to separate the pharmaceutically acceptable salt of Formula (I) or solvate (e.g., a hydrate) thereof from the solvent. In some embodiments, the purity of the pharmaceutically acceptable salt of Formula (I) or solvate (e.g., a hydrate) thereof after slurrying and separating is greater than 98%. In some embodiments, the purity of the pharmaceutically acceptable salt of Formula (I) or solvate (e.g., a hydrate) thereof after slurrying and separating is about 98%.

In some embodiments, the process comprises preparing the salt of Formula (II) by contacting a compound of Formula (III)

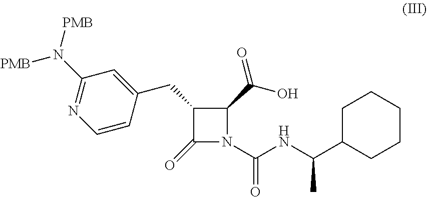

with trifluoroacetic acid.

In some embodiments, the process further comprises contacting the compound of Formula (III) with a silane (e.g., triethylsilane).

In some embodiments, the process produces over 500 grams of the compound of Formula (III) (e.g., over 1 kg).

In some embodiments, the process comprises preparing the compound of Formula (III) by contacting a compound of Formula (IV)

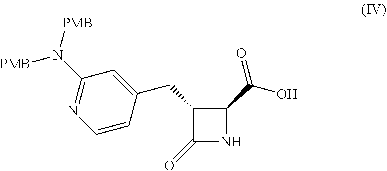

with a compound of Formula (V)

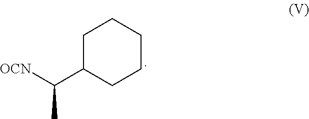

In some embodiments, process produces over 1 kilogram of the compound of Formula (III) (e.g., about 1.3 kg). In some embodiments, the process is carried out in the presence of a solvent. In some embodiments, the process is carried out in the presence of a base, (e.g., 1,8-diazabicyclo(5.4.0)undec-7-ene).

In some embodiments, the process comprises preparing a compound of Formula (IV) by contacting a compound of Formula (VI)

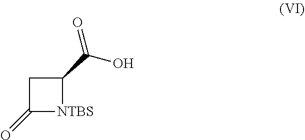

with the compound of Formula (VII)

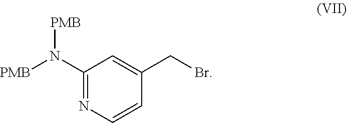

In some embodiments, the process produces over 500 grams of the compound of Formula (IV) (e.g., over 900 grams).

In some embodiments, the compound of Formula (III) is purified by a purification method that is not chromatography. In some embodiments, the purification method comprises slurrying the compound of Formula (III) in a solvent (e.g., heptane) and then filtering the compound of Formula (III) to separate the compound of Formula (III) from the solvent. In some embodiments, the purity of the compound of Formula (III) is greater than 90%.

In some embodiments, the compound of Formula (I) is purified by a purification method that is not chromatography.

### Methods of Treatment, Prophylaxis, or Reduction of Risk

The compounds described herein (e.g., Compound 1 or a pharmaceutically acceptable salt thereof) can inhibit Factor XIa or kallikrein. In some embodiments, a compound described herein can inhibit both Factor XIa and kallikrein. As a result, these compounds can be useful in the treatment, prophylaxis, or reduction in the risk of a disorder described herein.

Exemplary disorders include thrombotic events associated with coronary artery and cerebrovascular disease, venous or arterial thrombosis, coagulation syndromes, ischemia (e.g., coronary ischemia) and angina (stable and unstable), deep vein thrombosis (DVT), hepatic vein thrombosis, disseminated intravascular coagulopathy, Kasabach-Merritt syndrome, pulmonary embolism, myocardial infarction (e.g., ST-elevation myocardial infarction or non-ST-elevation myocardial infarction (e.g., non-ST-elevation myocardial infarction before catheterization), cerebral infarction, cerebral thrombosis, transient ischemic attacks, atrial fibrillation (e.g., non-valvular atrial fibrillation), cerebral embolism, thromboembolic complications of surgery (e.g., hip or knee replacement, orthopedic surgery, cardiac surgery, lung surgery, abdominal surgery, or endarterectomy) and peripheral arterial occlusion and may also be useful in treating or preventing myocardial infarction, stroke (e.g., large vessel acute ischemic stroke), angina and other consequences of atherosclerotic plaque rupture. The compounds of the invention possessing Factor XIa or kallikrein inhibition activity may also be useful in preventing thromboembolic disorders, e.g., venous thromboembolisms, in cancer patients, including those receiving chemotherapy and/or those with elevated lactase dehydrogenase (LDH) levels, and to prevent thromboembolic events at or following tissue plasminogen activator-based or mechanical restoration of blood vessel patency. The compounds of the invention possessing Factor XIa or kallikrein inhibition activity may also be useful as inhibitors of blood coagulation such as during the preparation, storage and fractionation of whole blood. Additionally, the compounds described herein may be used in acute hospital settings or periprocedurally, where a patient is at risk of a thromboembolic disorder or complication, and also in patients who are in a heightened coagulation state, e.g., cancer patients.

Factor XIa inhibition, according to the present invention, can be a more effective and safer method of inhibiting thrombosis compared to inhibiting other coagulation serine proteases such as thrombin or Factor Xa. Administration of a small molecule Factor XIa inhibitor should have the effect of inhibiting thrombin generation and clot formation with no or substantially no effect on bleeding times and little or no impairment of haemostasis. These results differ substantially from that of other “direct acting” coagulation protease inhibitors (e.g., active-site inhibitors of thrombin and Factor Xa), which demonstrate prolongation of bleeding time and less separation between antithrombotic efficacy and bleeding time prolongation. A preferred method according to the invention comprises administering to a mammal a pharmaceutical composition containing at least one compound of the invention.

The compounds described herein (e.g., Compound 1 or pharmaceutically acceptable salts thereof) can inhibit kallikrein. As a result, these compounds can be useful in the treatment, prophylaxis, or reduction in the risk of diseases involved in inflammation, such as edema (e.g., cerebral edema, macular edema, and angioedema (e.g., hereditary angioedema)). In some embodiments, the compounds of the invention can be useful in the treatment or prevention of hereditary angioedema. The compounds described herein (e.g., Compound 1) can also be useful in the treatment, prophylaxis, or reduction in the risk of, e.g., stroke, ischemia (e.g., coronary ischemia), and perioperative blood loss for example, Compound 1 or pharmaceutically acceptable salts thereof. The methods of the present invention are useful for treating or preventing those conditions which involve the action of Factor XIa or kallikrein. Accordingly, the methods of the present invention are useful in treating consequences of atherosclerotic plaque rupture including cardiovascular diseases associated with the activation of the coagulation cascade in thrombotic or thrombophilic states.

More particularly, the methods of the present invention can be used in the treatment, prophylaxis, or reduction in the risk of acute coronary syndromes such as coronary artery disease, myocardial infarction, unstable angina (including crescendo angina), ischemia (e.g., ischemia resulting from vascular occlusion), and cerebral infarction. The methods of the present invention further may be useful in the treatment, prophylaxis, or reduction in the risk of stroke (e.g., large vessel acute ischemic stroke) and related cerebral vascular diseases (including cerebrovascular accident, vascular dementia, and transient ischemic attack); venous thrombosis and thrombo-embolism, such as deep vein thrombosis (DVT) and pulmonary embolism; thrombosis associated with atrial fibrillation, ventricular enlargement, dilated cardiac myopathy, or heart failure; peripheral arterial disease and intermittent claudication; the formation of atherosclerotic plaques and transplant atherosclerosis; restenosis following arterial injury induced endogenously (by rupture of an atherosclerotic plaque), or exogenously (by invasive cardiological procedures such as vessel wall injury resulting from angioplasty or post-cranial artery stenting); disseminated intravascular coagulopathy, Kasabach-Merritt syndrome, cerebral thrombosis, and cerebral embolism.

Additionally, the methods of the present invention can be used in the treatment, prophylaxis (e.g., preventing), or reduction in the risk of thromboembolic consequences or complications associated with cancer, thrombectomy, surgery (e.g., hip replacement, orthopedic surgery), endarterectomy, introduction of artificial heart valves, peripheral vascular interventions (e.g., of the limbs), cerebrovascular interventions, large bore interventions used in the treatment of aneurysms, vascular grafts, mechanical organs, and implantation (e.g., trans-catheter aortic valve implantation) or transplantation of organs, (e.g., transplantation of the liver), tissue, or cells); percutaneous coronary interventions; catheter ablation; hemophilia therapy; hemodialysis; medications (such as tissue plasminogen activator or similar agents and surgical restoration of blood vessel patency) in patients suffering myocardial infarction, stroke (e.g., large vessel acute ischemic stroke), pulmonary embolism and like conditions; medications (such as oral contraceptives, hormone replacement, and heparin, e.g., for treating heparin-induced thrombocytopenia); sepsis (such as sepsis related to disseminated intravascular coagulation); pregnancy or childbirth; and another chronic medical condition. The methods of the present invention may be used to treat thrombosis due to confinement (e.g., immobilization, hospitalization, bed rest, or limb immobilization, e.g., with immobilizing casts, etc.). In some embodiments, the thromboembolic consequence or complication is associated with a percutaneous coronary intervention.

Additionally, the compounds described herein (e.g., Compound 1) or pharmaceutically acceptable salts thereof or compositions thereof can be useful in the treatment, prophylaxis and reduction in the risk of a thromboembolic disorder, e.g., a venous thromboembolism, deep vein thrombosis or pulmonary embolism, or associated complication in a subject, wherein the subject is exposed to an artificial surface. The artificial surface can contact the subject's blood, for example, as an extracorporeal surface or that of an implantable device. Such artificial surfaces include, but are not limited to, those of dialysis catheters, cardiopulmonary bypass circuits, artificial heart valves, e.g., mechanical heart valves (MHVs), ventricular assist devices, small caliber grafts, central venous catheters, extracorporeal membrane oxygenation (ECMO) apparatuses. Further, the thromboembolic disorder or associated complication may be caused by the artificial surface or associated with the artificial surface. For example, foreign surfaces and various components of mechanical heart valves (MHVs) are pro-thrombotic and promote thrombin generation via the intrinsic pathway of coagulation. Further, thrombin and FXa inhibitors are contraindicated with thromboembolic disorders or associated complications caused by artificial surfaces such as those MHVs, as these inhibitors are ineffective at blocking the intrinsic pathway at plasma levels that will not cause heavy bleeding. The compounds of the present invention, which can be used as, for example, Factor XIa inhibitors, are thus contemplated as alternative therapeutics for these purposes.

The compounds described herein (e.g., Compound 1) or pharmaceutically acceptable salts thereof or compositions thereof can also be useful for the treatment, prophylaxis, or reduction in the risk of atrial fibrillation in a subject in need thereof. For example, the subject can have a high risk of developing atrial fibrillation. The subject can also in need of dialysis, such as renal dialysis. The compounds described herein (e.g., Compound 1) or pharmaceutically acceptable salts thereof or compositions thereof can be administered before, during, or after dialysis. Direct oral anticoagulants (DOACs) currently available on the market, such as certain FXa or thrombin inhibitors, are contraindicated for atrial fibrillation under such a condition. The compounds of the present invention, which can be used as, for example, Factor XIa inhibitors, are thus contemplated as alternative therapeutics for these purposes. Additionally, the subject can be at a high risk of bleeding. In some embodiments, the subject can have end-stage renal disease. In other cases, the subject is not in need of dialysis, such as renal dialysis. Further, the atrial fibrillation can be associated with another thromboembolic disorder such as a blood clot.

Furthermore, the compounds described herein (e.g., Compound 1) or pharmaceutically acceptable salts thereof or compositions thereof can be used in the treatment, prophylaxis, or reduction in the risk of hypertension, e.g., arterial hypertension, in a subject. In some embodiments, the hypertension, e.g., arterial hypertension, can result in atherosclerosis. In some embodiments, the hypertension can be pulmonary arterial hypertension.

Furthermore, the compounds described herein (e.g., Compound 1) or pharmaceutically acceptable salts thereof or compositions thereof can be used in the treatment, prophylaxis, or reduction in the risk of disorders such as heparin-induced thrombocytopenia, heparin-induced thrombocytopenia thrombosis, or thrombotic microangiopathy, e.g., hemolytic uremic syndrome (HUS) or thrombotic thrombocytopenic purpura (TTP).

In some embodiments, the subject is sensitive to or has developed sensitivity to heparin. Heparin-induced thrombocytopenia (HIT) is the development of (a low platelet count), due to the administration of various forms of heparin. HIT is caused by the formation of abnormal antibodies that activate platelets. HIT can be confirmed with specific blood tests. In some embodiments, the subject is resistant to or has developed resistance to heparin. For example, activated clotting time (ACT) test can be performed on the subject to test for sensitivity or resistance towards heparin. The ACT test is a measure of the intrinsic pathway of coagulation that detects the presence of fibrin formation. A subject who is sensitive and/or resistant to standard dose of heparin typically do not reach target anticoagulation time. Common correlates of heparin resistance include, but are not limited to, previous heparin and/or nitroglycerin drips and decreased antithrombin III levels. In some embodiments, the subject has previously been administered an anticoagulant (e.g. bivalirudin/Angiomax).

The compounds described herein (e.g., Compound 1) or pharmaceutically acceptable salts thereof or compositions thereof can be used to reduce inflammation in a subject. In some embodiments, the inflammation can be vascular inflammation. In some embodiments, the vascular inflammation can be accompanied by atherosclerosis. In some embodiments, the vascular inflammation can be accompanied by a thromboembolic disease in the subject. In some embodiments, the vascular inflammation can be angiotensin II-induced vascular inflammation.

The compounds described herein (e.g., Compound 1) or pharmaceutically acceptable salts thereof or compositions thereof can be used in the treatment, prophylaxis, or reduction in the risk of renal disorders or dysfunctions, including end-stage renal disease, hypertension-associated renal dysfunction in a subject, kidney fibrosis, and kidney injury.

The methods of the present invention may also be used to maintain blood vessel patency, for example, in patients undergoing thrombectomy, transluminal coronary angioplasty, or in connection with vascular surgery such as bypass grafting, arterial reconstruction, atherectomy, vascular grafts, stent patency, and organ, tissue or cell implantation and transplantation. The inventive methods may be used to inhibit blood coagulation in connection with the preparation, storage, fractionation, or use of whole blood. For example, the inventive methods may be used in maintaining whole and fractionated blood in the fluid phase such as required for analytical and biological testing, e.g., for ex vivo platelet and other cell function studies, bioanalytical procedures, and quantitation of blood-containing components, or for maintaining extracorporeal blood circuits, as in a renal replacement solution (e.g., hemodialysis) or surgery (e.g., open-heart surgery, e.g., coronary artery bypass surgery). In some embodiments, the renal replacement solution can be used to treat patients with acute kidney injury. In some embodiments, the renal replacement solution can be continuous renal replacement therapy.

In addition, the methods of the present invention may be useful in treating and preventing the prothrombotic complications of cancer. The methods may be useful in treating tumor growth, as an adjunct to chemotherapy, for preventing angiogenesis, and for treating cancer, more particularly, cancer of the lung, prostate, colon, breast, ovaries, and bone.

### Extracorporeal Membrane Oxygenation (ECMO)

“Extracorporeal membrane oxygenation” (or “ECMO”) as used herein, refers to extracorporeal life support with a blood pump, artificial lung, and vascular access cannula, capable of providing circulatory support or generating blood flow rates adapted to support blood oxygenation, and optionally carbon dioxide removal. In venovenous ECMO, extracorporeal gas exchange is provided to blood that has been withdrawn from the venous system; the blood is then reinfused to the venous system. In venoarterial ECMO, gas exchange is provided to blood that is withdrawn from the venous system and then infused directly into the arterial system to provide partial or complete circulatory or cardiac support. Venoarterial ECMO allows for various degrees of respiratory support.

As used herein, “extracorporeal membrane oxygenation” or “ECMO” refers to extracorporeal life support that provides circulatory support or generates blood flow rates adequate to support blood oxygenation. In some embodiments, ECMO comprises removal of carbon dioxide from a subject's blood. In some embodiments, ECMO is performed using an extracorporeal apparatus selected from the group consisting of a blood pump, artificial lung, and vascular access cannula.

As used herein, “venovenous ECMO” refers to a type of ECMO in which blood is withdrawn from the venous system of a subject into an ECMO apparatus and subjected to gas exchange (including oxygenation of the blood), followed by reinfusion of the withdrawn blood into the subject's venous system. As used herein, “venoarterial ECMO” refers to a type of ECMO in which blood is withdrawn from the venous system of a subject into an ECMO apparatus and subjected to gas exchange (including oxygenation of the blood), followed by infusion of the withdrawn blood directly into the subject's arterial system. In some embodiments, venoarterial ECMO is performed to provide partial circulatory or cardiac support to a subject in need thereof. In some embodiments, venoarterial ECMO is performed to provide complete circulatory or cardiac support to a subject in need thereof.

The compounds of the present invention can be used in the treatment, prophylaxis, or reduction in the risk of a thromboembolic disorder in a subject in need thereof, wherein the subject is exposed to an artificial surface such as that of an extracorporeal membrane oxygenation (ECMO) apparatus (vide supra), which can be used as a rescue therapy in response to cardiac or pulmonary failure. The surface of an ECMO apparatus that directly contacts the subject can be a pro-thrombotic surface that can result in a thromboembolic disorder such as a venous thromboembolism, e.g., deep vein thrombosis or pulmonary embolism, leading to difficulties in treating a patient in need of ECMO. Clots in the circuit are the most common mechanical complication (19%). Major clots can cause oxygenator failure, and pulmonary or systemic emboli.

ECMO is often administered with a continuous infusion of heparin as an anticoagulant to counter clot formation. However, cannula placement can cause damage to the internal jugular vein, which causes massive internal bleeding. Bleeding occurs in 30-40% of patients receiving ECMO and can be life-threatening. This severe bleeding is due to both the necessary continuous heparin infusion and platelet dysfunction. Approximately 50% of reported deaths are due to severe bleeding complications. Aubron et al. Critical Care, 2013, 17:R73 looked at the factors associated with ECMO outcomes.

The compounds of the present invention, which can be used as, for example, Factor XIa inhibitors, are thus contemplated as an alternative replacement for heparin in ECMO therapy. The compounds of the present invention are contemplated as effective agents for blocking the intrinsic pathway at plasma levels that will afford effective anti-coagulation/anti-thrombosis without marked bleeding liabilities. In some embodiments, the subject is sensitive to or has developed sensitivity to heparin. In some embodiments, the subject is resistant to or has developed resistance to heparin.

### Ischemia

“Ischemia” or an “ischemic event” is a vascular disease generally involving vascular occlusion or a restriction in blood supply to tissues. Ischemia can cause a shortage of oxygen and glucose needed for cellular metabolism. Ischemia is generally caused by problematic blood vessels that result in damage or dysfunction of tissue. Ischemia can also refer to a local loss in blood or oxygen in a given part of the body resulting from congestion (e.g., vasoconstriction, thrombosis, or embolism). Causes include embolism, thrombosis of an atherosclerosis artery, trauma, venous problems, aneurysm, heart conditions (e.g., myocardial infarction, mitral valve disease, chronic arterial fibrillation, cardiomyopathies, and prosthesis), trauma or traumatic injury (e.g., to an extremity producing partial or total vessel occlusion), thoracic outlet syndrome, atherosclerosis, hypoglycemia, tachycardia, hypotension, outside compression of a blood vessel (e.g., by a tumor), sickle cell disease, localized extreme cold (e.g., by frostbite), tourniquet application, glutamate receptor stimulation, arteriovenous malformations, rupture of significant blood vessels supplying a tissue or organ, and anemia.

A transient ischemic event generally refers to a transient (e.g., short-lived) episode of neurologic dysfunction caused by loss of blood flow (e.g., in the focal brain, spinal cord, or retinal) without acute infarction (e.g., tissue death). In some embodiments, the transient ischemic event lasts for less than 72 hours, 48 hours, 24 hours, 12 hours, 10 hours, 8 hours, 4 hours, 2 hours, 1 hour, 45 minutes, 30 minutes, 20 minutes, 15 minutes, 10 minutes, 5 minutes, 4 minutes, 3 minutes, 2 minutes, or 1 minute.

### Angioedema

Angioedema is the rapid swelling of the dermis, subcutaneous tissue, mucosa, and submucosal tissues. Angioedema is typically classified as either hereditary or acquired.

“Acquired angioedema” can be immunologic, non-immunologic, or idiopathic; caused by e.g., allergy, as a side effect of medications, e.g., ACE inhibitor medications.

“Hereditary angioedema” or “HAE” refers to a genetic disorder that results in acute periods of edema (e.g., swelling) that may occur in nearly all parts of the body, including the face, limbs, neck, throat, larynx, extremities, gastrointestinal tract, and genitalia. Attacks of HAE can often be life-threatening, with severity depending on the area affected, e.g., abdominal attacks may result in intestinal obstruction, while swelling of the larynx and upper airway can lead to asphyxiation. Pathogenesis of hereditary angioedema may be related to unopposed activation of the contact pathway by the initial generation of kallikrein or clotting factors (e.g., Factor XII).

Signs and symptoms include swelling, e.g., of the skill of the face, mucosa of the mouth or throat, and tongue. Itchiness, pain, decreased sensation in the affected areas, urticaria (i.e., hives), or stridor of the airway may also be a sign of angioedema. However, there can be no associated itch, or urticaria, e.g., in hereditary angioedema. HAE subjects can experience abdominal pain (e.g., abdominal pain lasting one to five days, abdominal attacks increasing a subject's white blood cell count), vomiting, weakness, watery diarrhea, or rash.

Bradykinin plays an important role in angioedema, particularly hereditary angioedema. Bradykinin is released by various cell types in response to numerous different stimuli and is a pain mediator. Interfering with bradykinin production or degradation can lead to angioedema. In hereditary angioedema, continuous production of enzyme kallikrein can facilitate bradykinin formation. Inhibition of kallikrein can interfere with bradykinin production; and treat or prevent angioedema.

The methods described herein can include those in which a subject's blood is in contact with an artificial surface. For example, in an aspect, provided herein is a method of treating a thromboembolic disorder in a subject in need thereof, the method comprising administering to the subject an effective amount of a compound represented by

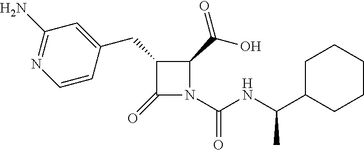 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In an aspect, provided herein is a method of reducing the risk of a thromboembolic disorder in a subject in need thereof, the method comprising administering to the subject an effective amount of a compound represented by

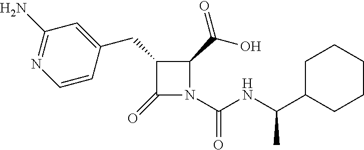 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In an aspect, provided herein is a method of prophylaxis of a thromboembolic disorder in a subject in need thereof, the method comprising administering to the subject an effective amount of a compound represented by

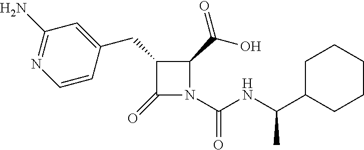 [SMILES: C[C@@H](NC(=O)N1C(=O)[C@H](Cc2ccnc(N)c2)[C@H]1C(=O)O)C1CCCCC1]

or a pharmaceutically acceptable salt thereof, wherein the blood of the subject is contacted with an artificial surface.

In some embodiments of the methods provided herein, the artificial surface is in contact with blood in the subject's circulatory system. In some embodiments, the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus. In some embodiments, the artificial surface causes or is associated with the thromboembolic disorder. In some embodiments, the thromboembolic disorder is a venous thromboembolism, deep vein thrombosis, or pulmonary embolism. In some embodiments, the thromboembolic disorder is a blood clot.

In some embodiments, the methods further comprise conditioning the artificial surface with a separate dose of the compound or pharmaceutically acceptable salt thereof, prior to contacting the artificial surface with blood in the circulatory system of the subject. In some embodiments, the methods further comprise conditioning the artificial surface with a separate dose of the compound or pharmaceutically acceptable salt thereof prior to or during administration of the compound or a pharmaceutically acceptable salt thereof to the subject. In some embodiments, the methods further comprise conditioning the artificial surface with a separate dose of the compound or pharmaceutically acceptable salt thereof prior to and during administration of the compound or a pharmaceutically acceptable salt thereof to the subject.

In an aspect, provided herein is a method of treating the blood of a subject in need thereof, the method comprising administering to the subject an effective amount of a compound represented by

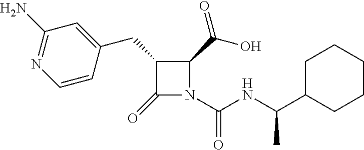 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In an aspect, provided herein is a method of maintaining the plasma level of a compound represented by

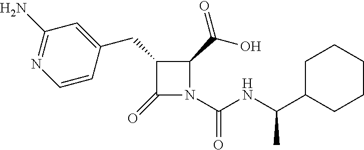 [SMILES: C[C@@H](NC(=O)N1C(=O)[C@H](Cc2ccnc(N)c2)[C@H]1C(=O)O)C1CCCCC1]

or a pharmaceutically acceptable salt thereof, in the blood of a subject in contact with an artificial surface, the method comprising:

(i) administering the compound or pharmaceutically acceptable salt thereof to the subject prior to or while contacting the artificial surface with the blood of the subject; and

(ii) conditioning an artificial surface with the compound or a pharmaceutically acceptable salt thereof prior to or while contacting the artificial surface with the blood of the subject;

thereby maintaining the plasma level of the compound or a pharmaceutically acceptable salt thereof in the blood of the subject.

In some embodiments of the methods described herein, the compound, or a pharmaceutically acceptable salt thereof, maintains a constant activated partial thromboplastin time (aPTT) in the blood of the subject before and after contact with the artificial surface. In some embodiments, the compound or a pharmaceutically acceptable salt thereof is administered to the subject prior to and while contacting the artificial surface with the blood of the subject.

In some embodiments, the artificial surface is conditioned with the compound or a pharmaceutically acceptable salt thereof prior to and while contacting the artificial surface with the blood of the subject. In some embodiments, the method further prevents or reduces risk of a blood clot formation in the blood of the subject in contact with the artificial surface.

In some embodiments, the artificial surface is a cardiopulmonary bypass circuit. In some embodiments, the artificial surface is an extracorporeal membrane oxygenation (ECMO) apparatus. In some embodiments, the ECMO apparatus is venovenous ECMO apparatus or venoarterial ECMO apparatus.

In an aspect, provided herein is a method of preventing or reducing a risk of a thromboembolic disorder in a subject during or after a medical procedure, comprising:

(i) administering to the subject an effective amount of a compound represented by:

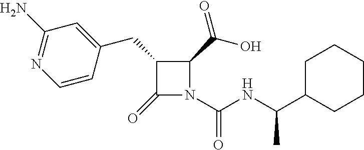 [SMILES: C[C@@H](NC(=O)N1C(=O)[C@H](Cc2ccnc(N)c2)[C@H]1C(=O)O)C1CCCCC1]

or pharmaceutically acceptable salt thereof, before, during, or after the medical procedure; and

(ii) contacting blood of the subject with an artificial surface;

thereby preventing or reducing the risk of the thromboembolic disorder during or after the medical procedure.

In some embodiments, the artificial surface is conditioned with the compound or pharmaceutically acceptable salt thereof prior to administration of the compound to the subject prior to, during, or after the medical procedure.

In some embodiments, the artificial surface is conditioned with a solution comprising the compound or a pharmaceutically acceptable salt thereof prior to administration of the compound or a pharmaceutically acceptable salt thereof to the subject prior to, during, or after the medical procedure. In some embodiments, the solution is a saline solution, Ringer's solution, or blood. In some embodiments, the solution further comprises blood. In some embodiments, the blood is acquired from the subject or a donor.

In some embodiments, the thromboembolic disorder is a blood clot.

In some embodiments, the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

In some embodiments, the medical procedure comprises a cardiopulmonary bypass. In some embodiments, the medical procedure comprises an oxygenation and pumping of blood via extracorporeal membrane oxygenation (ECMO). In some embodiments, the ECMO is venovenous ECMO or venoarterial ECMO.

In some embodiments of the methods described herein, the pharmaceutically acceptable salt of the compound is a hydrochloride salt. In some embodiments, the subject is a human. In some embodiments, the subject has an elevated risk of a thromboembolic disorder. In some embodiments, the thromboembolic disorder is a result of a complication in surgery.

In some embodiments, the subject is sensitive to or has developed sensitivity to heparin. In some embodiments, the subject is resistant to or has developed resistance to heparin.

In some embodiments, the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

### Pharmaceutical Compositions

The compositions described herein include the compound described herein (e.g., Compound 1 as well as additional therapeutic agents, if present, in amounts effective for achieving the treatment of a disease or disease symptoms (e.g., such as a disease associated with Factor XIa or kallikrein).

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions may be in the form of a solid lyophilized composition that can be reconstituted by addition of a compatible reconstitution diluent prior to parenteral administration or in the form of a frozen composition adapted to be thaws and, if desired, diluted with a compatible diluent prior to parenteral administration. In some embodiments, the pharmaceutical composition includes a powder (e.g., lyophilized composition) dissolved in aqueous medium, e.g., a saline solution, in a unit dosage IV bag or bottle at a concentration suitable for intravenous administration to a subject. In some embodiments, ingredients of a pharmaceutical composition suitable for intravenous administration are separated from each other in a single container, e.g., a powder comprising a compound described herein or a pharmaceutically acceptable salt thereof, is separated from an aqueous medium such as a saline solution. In this latter example, the various components are separated by a seal that can be broken to contact the ingredients with each other to form the pharmaceutical composition suitable for intravenous administration.

### Routes of Administration

The pharmaceutical compositions provided herewith may be administered orally, rectally, or parenterally (e.g., intravenous infusion, intravenous bolus injection, inhalation, implantation). The term parenteral as used herein includes subcutaneous, intracutaneous, intravenous (e.g., intravenous infusion, intravenous bolus injection), intranasal, inhalation, pulmonary, transdermal, intramuscular, intraarticular, intraarterial, intrasynovial, intrasternal, intrathecal, intralesional and intracranial injection or other infusion techniques. The pharmaceutical compositions provided herewith may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The pharmaceutical compositions may be in the form of a sterile injectable preparation, for example, as a sterile injectable aqueous or oleaginous solution or suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans or other similar emulsifying agents or bioavailability enhancers which are commonly used in the manufacture of pharmaceutically acceptable solid, liquid, or other dosage forms may also be used for the purposes of formulation. In some embodiments, the intravenous pharmaceutical composition comprises a carrier selected from the group consisting of 5% w/w dextrose water (“5DW”) and saline.

The pharmaceutical compositions provided herewith may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening or flavoring or coloring or taste masking agents may be added.

The compounds described herein can, for example, be administered by injection, intravenously (e.g., intravenous infusion, intravenous bolus injection), intraarterially, subdermally, intraperitoneally, intramuscularly, or subcutaneously; or orally, buccally, nasally, transmucosally, topically with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day (e.g., by intravenous bolus injection) or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

In some embodiments, a pharmaceutical composition formulated for oral administration, subcutaneous administration, or intravenous administration is administered to a subject from 1 time per day to 6 times per day (e.g., 2 times per day or 4 times per day). In some embodiments, a pharmaceutical composition formulated for oral administration is administered to a subject from 1 time per day to 6 times per day (e.g., 2 times per day or 4 times per day) for about 3 to 9 months. In some embodiments, a pharmaceutical composition formulated for oral administration is administered to a subject from 1 time per day to 6 times per day (e.g., 2 times per day or 4 times per day) for about 1 year. In some embodiments, a pharmaceutical composition formulated for oral administration is administered to a subject from 1 time per day to 6 times per day (e.g., 2 times per day or 4 times per day) for the rest of his or her life.

In some embodiments, the compound or pharmaceutical composition is administered to the subject intravenously. In some embodiments, the compound or pharmaceutical composition is administered to the subject subcutaneously. In some embodiments, the compound or pharmaceutical composition is administered to the subject as a continuous intravenous infusion. In some embodiments, the compound or pharmaceutical composition is administered to the subject as a bolus. In some embodiments, the compound or pharmaceutical composition is administered to the subject as a bolus followed by a continuous intravenous infusion.

### Combinations

In carrying out the methods of the present invention, it may be desired to administer the compounds of the invention (e.g., Factor XIa or kallikrein inhibitors) in combination with each other and one or more other agents for achieving a therapeutic benefit such as antithrombotic or anticoagulant agents, anti-hypertensive agents, anti-ischemic agents, anti-arrhythmic agents, platelet function inhibitors, and so forth. For example, the methods of the present invention may be carried out by administering the small molecule Factor XIa or kallikrein inhibitors in combination with a small molecule Factor XIa or kallikrein inhibitor. More particularly, the inventive methods may be carried out by administering the small molecule Factor XIa or kallikrein inhibitors in combination with aspirin, clopidogrel, ticlopidine or CS-747, warfarin, low molecular weight heparins (such as LOVENOX), GPIIb/GPIIIa blockers, PAI-1 inhibitors such as XR-330 and T-686, P2Y1 and P2Y12 receptor antagonists; thromboxane receptor antagonists (such as ifetroban), prostacyclin mimetics, thromboxane A synthetase inhibitors (such as picotamide), serotonin-2-receptor antagonists (such as ketanserin); compounds that inhibit other coagulation factors such as FVII, FVIII, FIX, FX, prothrombin, TAFI, and fibrinogen, or other compounds that inhibit FXI or kallikrein; fibrinolytics such as TPA, streptokinase, PAI-1 inhibitors, and inhibitors of α-2-antiplasmin such as anti-α-2-antiplasmin antibody fibrinogen receptor antagonists, inhibitors of α-1-antitrypsin, hypolipidemic agents, such as HMG-CoA reductase inhibitors (e.g., pravastatin, simvastatin, atorvastatin, fluvastatin, cerivastatin, AZ4522, and itavastatin), and microsomal triglyceride transport protein inhibitors (such as disclosed in U.S. Pat. Nos. 5,739,135, 5,712,279 and 5,760,246); antihypertensive agents such as angiotensin-converting enzyme inhibitors (e.g., captopril, lisinopril or fosinopril); angiotensin-II receptor antagonists (e.g., irbesartan, losartan or valsartan); ACE/NEP inhibitors (e.g., omapatrilat and gemopatrilat); or β-blockers (such as propranolol, nadolol and carvedilol). The inventive methods may be carried out by administering the small molecule Factor XIa or kallikrein inhibitors in combination with anti-arrhythmic agents such as for atrial fibrillation, for example, amiodarone or dofetilide. The inventive methods may also be carried out in combination continuous renal replacement therapy for treating, e.g., acute kidney injury.

In carrying out the methods of the present invention, it may be desired to administer the compounds of the invention (Factor XIa or kallikrein inhibitors) in combination with agents that increase the levels of cAMP or cGMP in cells for a therapeutic benefit. For example, the compounds of the invention may have advantageous effects when used in combination with phosphodiesterase inhibitors, including PDE1 inhibitors (such as those described in Journal of Medicinal Chemistry, Vol. 40, pp. 2196-2210 [1997]), PDE2 inhibitors, PDE3 inhibitors (such as revizinone, pimobendan, or olprinone), PDE4 inhibitors (such as rolipram, cilomilast, or piclamilast), PDE7 inhibitors, or other PDE inhibitors such as dipyridamole, cilostazol, sildenafil, denbutyline, theophylline (1,2-dimethylxanthine), ARIFLOT™ (i.e., cis-4-cyano-4-[3-(cyclopentylox-y)-4-methoxyphenyl]cyclohexane-1-carboxyl-ic acid), arofyline, roflumilast, C-11294A, CDC-801, BAY-19-8004, cipamfylline, SCH351591, YM-976, PD-189659, mesiopram, pumafentrine, CDC-998, IC-485, and KW-4490.

The inventive methods may be carried out by administering the compounds of the invention in combination with prothrombolytic agents, such as tissue plasminogen activator (natural or recombinant), streptokinase, reteplase, activase, lanoteplase, urokinase, prourokinase, anisolated streptokinase plasminogen activator complex (ASPAC), animal salivary gland plasminogen activators, and the like.

The inventive methods may be carried out by administering the compounds of the invention in combination with β-adrenergic agonists such as albuterol, terbutaline, formoterol, salmeterol, bitolterol, pilbuterol, or fenoterol; anticholinergics such as ipratropium bromide; anti-inflammatory cortiocosteroids such as beclomethasone, triamcinolone, budesonide, fluticasone, flunisolide or dexamethasone; and anti-inflammatory agents such as cromolyn, nedocromil, theophylline, zileuton, zafirlukast, monteleukast and pranleukast.

Small molecule Factor XIa or kallikrein inhibitors may act synergistically with one or more of the above agents. Thus, reduced doses of thrombolytic agent(s) may be used, therefore obtaining the benefits of administering these compounds while minimizing potential hemorrhagic and other side effects.

### Course of Treatment

The compositions described herein include an effective amount of a compound of the invention (e.g., a Factor XIa or kallikrein inhibitor) in combination and one or more other agents (e.g., an additional therapeutic agent) such as antithrombotic or anticoagulant agents, anti-hypertensive agents, anti-ischemic agents, anti-arrhythmic agents, platelet function inhibitors, and so forth for achieving a therapeutic benefit.

In some embodiments, the additional therapeutic agent is administered following administration of the compound of the invention (e.g., a Factor XIa or kallikrein inhibitor).

In some embodiments, the additional therapeutic agent is administered 15 minutes, 30 minutes, 1 hour, 2 hours, 4 hours, 6 hours, 8 hours, 10 hours, 12 hours, 14 hours, 18 hours, 24 hours, 48 hours, 72 hours or longer after administration of the compound of the invention (e.g., a Factor XIa or kallikrein inhibitor). In some embodiments, the additional therapeutic agent is administered (e.g., orally) after discharge from a medical facility (e.g., a hospital).

In some embodiments, the compound of the invention (e.g., a Factor XIa or kallikrein inhibitor) and the additional therapeutic agent are co-formulated into a single composition or dosage. In some embodiments, the compound of the invention (e.g., a Factor XIa or kallikrein inhibitor) and the additional therapeutic agent are administered separately. In some embodiments, the compound of the invention (e.g., a Factor XIa or kallikrein inhibitor) and the additional therapeutic agent are administered sequentially. In some embodiments, the compound of the invention (e.g., a Factor XIa or kallikrein inhibitor) and the additional therapeutic agent are administered separately and sequentially. In general, at least one of the compound of the invention (e.g., a Factor XIa or kallikrein inhibitor) and the additional therapeutic agent is administered parenterally (e.g., intranasally, intramuscularly buccally, inhalation, implantation, transdermal, intravenously (e.g., intravenous infusion, intravenous bolus injection), subcutaneous, intracutaneous, intranasal, pulmonary, transdermal, intraarticular, intraarterial, intrasynovial, intrasternal, intrathecal, intralesional and intracranial injection or other infusion techniques); orally; or rectally, for example, intramuscular injection or intravenously (e.g., intravenous infusion, intravenous bolus injection)). In some embodiments, compound of the invention is administered parenterally (e.g., intranasally, buccally, intravenously (e.g., intravenous infusion, intravenous bolus injection) or intramuscularly). In some embodiments, the additional therapeutic agent is administered orally. In some embodiments, the compound of the invention (e.g., a Factor XIa or kallikrein inhibitor) is administered parenterally (e.g., intranasally, buccally, intravenously (e.g., intravenous infusion, intravenous bolus injection) or intramuscularly) and the additional therapeutic agent is administered orally.

In some embodiments, the compound of the invention (e.g., a Factor XIa or kallikrein inhibitor) may be administered once or several times a day. A duration of treatment may follow, for example, once per day for a period of about 1, 2, 3, 4, 5, 6, 7 days or more. In some embodiments, the treatment is chronic (e.g., for a lifetime). In some embodiments, either a single dose in the form of an individual dosage unit or several smaller dosage units or by multiple administrations of subdivided dosages at certain intervals is administered. For instance, a dosage unit can be administered from about 0 hours to about 1 hr, about 1 hr to about 24 hr, about 1 to about 72 hours, about 1 to about 120 hours, or about 24 hours to at least about 120 hours post injury. Alternatively, the dosage unit can be administered from about 0.5, 1, 1.5, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. Subsequent dosage units can be administered any time following the initial administration such that a therapeutic effect is achieved. In some embodiments, the initial dose is administered orally. In some embodiments, doses subsequent to the initial dose are administered parenterally (e.g., intranasally, intramuscularly buccally, inhalation, implantation, transdermal, intravenously (e.g., intravenous infusion, intravenous bolus injection), subcutaneous, intracutaneous, intranasal, pulmonary, transdermal, intraarticular, intraarterial, intrasynovial, intrasternal, intrathecal, intralesional and intracranial injection or other infusion techniques); orally; or rectally.

In some embodiments, compounds of the invention (e.g., a Factor XIa or kallikrein inhibitor) is administered orally, e.g., as an liquid or solid dosage form for ingestion, for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the compound of the invention (e.g., a Factor XIa or kallikrein inhibitor) is administered orally as a liquid dosage form. In another embodiment, the compound of the invention (e.g., a Factor XIa or kallikrein inhibitor) is administered orally as a solid dosage form.

Where a subject undergoing therapy exhibits a partial response, or a relapse following completion of the first cycle of the therapy, subsequent courses of therapy may be needed to achieve a partial or complete therapeutic response (e.g., chronic treatment, e.g., for a lifetime).

In some embodiments, the compound of the invention (e.g., a Factor XIa or kallikrein inhibitor) is administered intravenously, e.g., as an intravenous infusion or intravenous bolus injection, for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the compound of the invention (e.g., a Factor XIa or kallikrein inhibitor) is administered as an intravenous infusion for about 5, 10, 15, 30, 45, or 60 minutes or longer; about 1, 2, 4, 6, 8, 10, 12, 16, or 24 hours or longer; about 1, 2, 3, 4, 5, 6, 7, 8, 9, or 10 days or longer.

### Dosages and Dosing Regimens

The effective amount of a small molecule Factor XIa or kallikrein inhibitor administered according to the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### EXAMPLES

Starting materials and various intermediates described in the following examples may be obtained from commercial sources, prepared from commercially available organic compounds, or prepared using known synthetic methods. The examples set forth below are non-limiting in scope and include certain methods of preparing intermediates and final products, including their respective methods of purification.

### General Procedures

All non-aqueous reactions were run under an atmosphere of nitrogen to maintain an anhydrous atmosphere and to maximize yields. All reactions were stirred using an overhead stirring assembly or magnetically, with the aid of a Teflon-coated stir bar. The description ‘drying over’ refers to drying of a reaction product solution over a specified drying agent and then filtration of the solution though a suitable filter paper or through a sintered glass funnel. The descriptions ‘was concentrated’, ‘was concentrated at reduced pressure’, or ‘evaporated’ refers to removal of solvents under reduced pressure using a rotary evaporator. Chromatography or chromatographed refers to the use of flash column chromatography on silica gel unless otherwise specified. Flash chromatography refers to column chromatography under gas pressure (for example, nitrogen) or a mechanical pump to apply solvent pressure such as with a commercial system as supplied by Biotage or other vendors. Unless otherwise specified, proton NMR spectra (1H) are measured at 400 MHz and carbon NMR spectra (13C) are measured at 100 MHz in the specified solvent.

Abbreviations used in the experimental examples are listed in the Abbreviations Table below.

Abbreviation Table

| | |
|---|---|
| ACN | Acetonitrile |
| Celite ® | Diatomaceous earth |
| DBU | 1,8-Diazabicyclo(5.4.0)undec-7-ene |
| DCM | Dichloromethane |
| EA | Ethyl acetate |
| H | Hours |
| LC | HPLC |
| IPA | Isopropyl alcohol |
| LDA | Lithium diisopropylamide |
| Min | Minutes |
| MTBE | Methyl tert-butyl ether |
| NMR | Nuclear magnetic resonance instrument |
| PMB | 4-Methoxybenzyl |
| RT | Room temperature |
| TFA | Trifluoroacetic acid |
| TLC | Thin layer chromatography |
| THF | Tetrahydrofuran |
| Chromatography or | Purification of products using flash column |
| chromatographed | chromatography on silica gel |
| Concentrated or | Concentration of organic solutions under |
| concentrated in | reduced pressure with the use of a rotary |
| vacuo | evaporator |

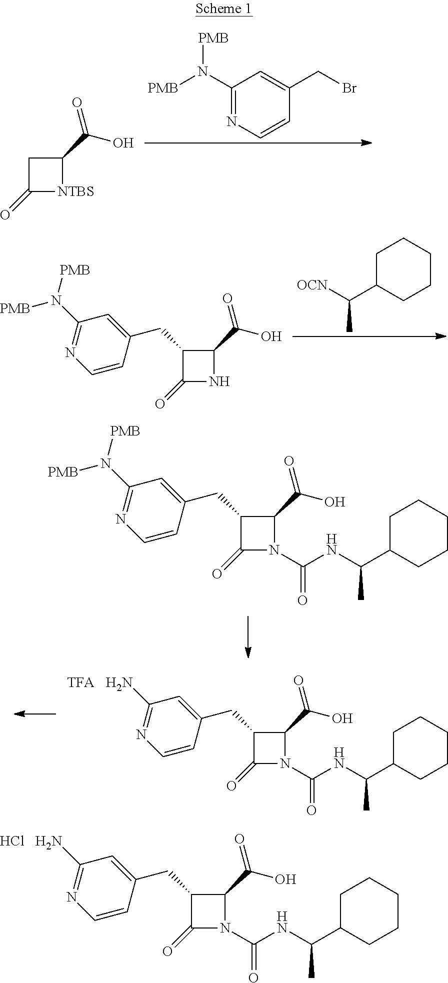

Scheme 1 illustrates a general method for the preparation of Compound 1.HCl.

### Example 1

### Preparation of Intermediate (R)-(1-isocyanatoethyl)cyclohexane

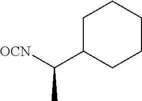

(R)-1-cyclohexylethanamine was dissolved in DCM and aqueous NaHCO3 solution was added. The heterogeneous mixture was cooled to −2° C. and treated with triphosgene over about 6 h while maintaining the reaction temperature below 5° C. Water was added to the mixture and the phases were separated. The aqueous phase was back extracted twice with DCM. The combined DCM phases were concentrated to give a residue. The residue was treated with heptane and cooled to 5-10° C. to give a precipitate. The precipitate was collected and dried to give the title compound in 74% yield.

Below is an exemplary procedure of Example 1.

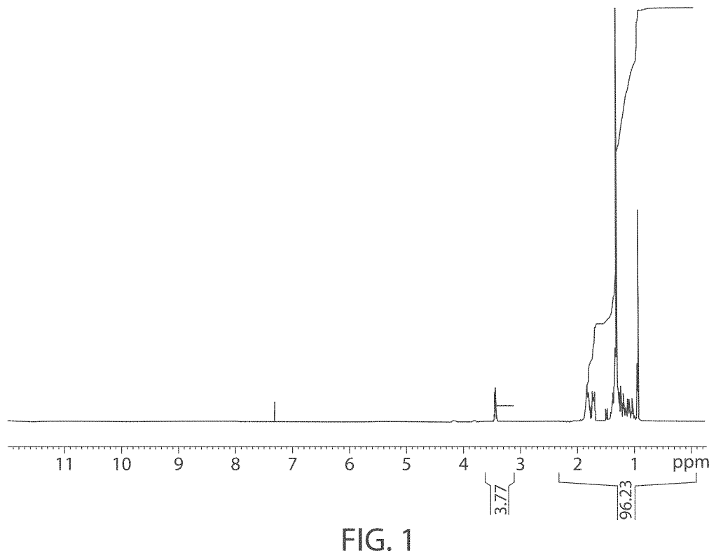

To a solution of (R)-1-cyclohexylethanamine (0.50 kg, 3.93 mol) in DCM (10.0 L) was added 9% aqueous NaHCO3 solution (10.0 L), and the mixture was cooled to 0° C. Triphosgene (0.38 kg, 1.30 mol) was added to the mixture while maintaining the reaction temperature and agitated for 1 h at 0° C. The reaction was monitored by TLC (100% EA eluent). Water (10 L) was added to the mixture and the phases were separated. The aqueous layer was extracted with DCM (2×5.0 L). The organic phase was concentrated to dryness without exceeding 45° C. Heptane (2×1 L) was added to the residue and the mixture was concentrated to give a solid. The solid was taken up in heptane (6.0 L), dried over MgSO4, rinsed with heptane (0.5 L) and concentrated to afford (R)-(1-isocyanatoethyl)cyclohexane. Exemplary yields are given in Table 1. NMR spectrum of Sample #4 dissolved in CDCl₃ is depicted in FIG. 1.

TABLE 1
Exemplary yields for the
preparation of (R)-(1-isocyanatoethyl)cyclohexane

| | | | | Confirmed by |
|---|---|---|---|---|
| Sample # | Charge, kg | Yield, g | Yield, % | NMR |
| 1 | 0.50 | 326.0 | 54.3 | Yes |
| 2 | 0.50 | 384.9 | 64.2 | Yes |
| 3 | 0.50 | 175.0 | 29.2 | Yes |
| 4 | 0.50 | 377.3 | 62.9 | Yes |
| 5 | 0.50 | 412.0 | 68.7 | Yes |
| 6 | 0.50 | 331.2 | 55.2 | Yes |
| 7 | 0.50 | 337.5 | 56.3 | Yes |

### Example 2

### Preparation of 4-(bromomethyl)-N,N-bis(4-methoxybenzyl)pyridin-2-amine

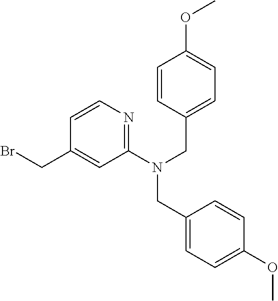 [SMILES: COc1ccc(CN(Cc2ccc(OC)cc2)c2cc(CBr)ccn2)cc1]

### Step 1. Preparation of 2-[Bis-(4-methoxy-benzyl)-amino]-isonicotinic acid methyl ester

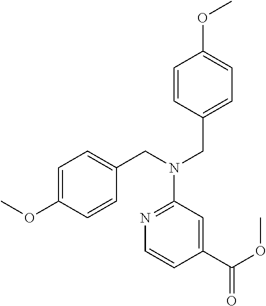

The mixture of 2-amino pyridine-4-carboxylic acid methyl ester (5.5 kg) and 4-methoxybenzyl chloride (14.64 kg) in 33 L of acetonitrile was heated to reflux for 3 h, then 7.3 kg of Et3N were added slowly into the refluxing mixture; then the reaction was cooled to room temperature and kept stirring overnight. After removal the acetonitrile and Et3N, a large amount of water was added into the mixture, thus precipitating a solid. After centrifugal operation and recrystallization of the solid in 5-10 L isopropanol, the reaction afforded 2-[bis-(4-methoxy-benzyl)-amino]-isonicotinic acid methyl ester (3 kg, 21%) as white solid.

### Step 2. Preparation of {2-[Bis-(4-methoxy-benzyl)-amino]-pyridin-4-yl}-methanol

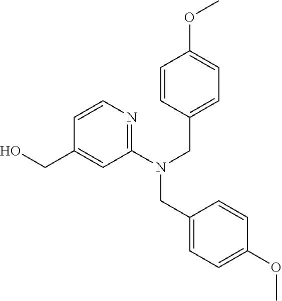 [SMILES: COc1ccc(CN(Cc2ccc(OC)cc2)c2cc(CO)ccn2)cc1]

To a 50 L reaction kettle, LiAlH4 (388 g) was added into 16 L of dry THF (0° C.) in batches. Then, 4 kg of 2-[bis-(4-methoxy-benzyl)-amino]-isonicotinic acid methyl ester in THF (16 L) was added into the mixture dropwise, while maintaining the reaction temperature at −5° C. Ethyl acetate (900 g), water (388 g) and 15% NaOH aqueous solution (388 g) were slowly added into the reaction mixture successively. After 10 min of stirring, anhydrous Na2SO4 (1.3 kg) was added to the mixture, and the corresponding mixture was stirred for 30 min. The mixture was vacuum filtered, and the filter cake was washed with THF (12 L), and then filtered. The combined filtrate was concentrated. The resulting solution that contains {2-[bis-(4-methoxy-benzyl)-amino]-pyridin-4-yl}-methanol was used directly in the next step without purification.

### Step 3. Preparation of 4-(bromomethyl)-N,N-bis(4-methoxybenzyl)pyridin-2-amine

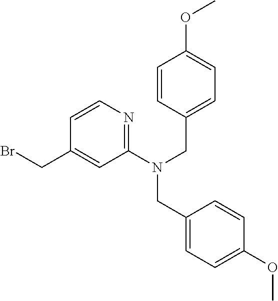 [SMILES: COc1ccc(CN(Cc2ccc(OC)cc2)c2cc(CBr)ccn2)cc1]

{2-[Bis-(4-methoxy-benzyl)-amino]-pyridin-4-yl}-methanol (5 kg) and CBr4 (5 kg) were added into 25 L of DCM, and the reaction solution was kept at 0-10° C. Then a solution of PPh₃ (4.32 kg) in 10 L of DCM was added to the reaction solution dropwise. The reaction was tracked by TLC, and if {2-[bis-(4-methoxy-benzyl)-amino]-pyridin-4-yl}-methanol was not completely consumed; PPh₃ was added into the reaction mixture until {2-[bis-(4-methoxy-benzyl)-amino]-pyridin-4-yl}-methanol was totally consumed. After removal of the DCM, the reaction afforded an oil-like product. This oil was stirred in 50% EtOH aqueous solution (16 L) at room temperature for 1 h. Then, the mixture was filtered, and the filter cake was washed with a small quantity of 50% EtOH aqueous solution. The residual filter cake was then stirred in 50% EtOH aqueous solution (8 L) at room temperature for another 1 h, filtered, and dried to afford the crystalline solid product (5.2 kg). ¹H NMR (CDCl₃) δ 3.78 (s, 6H), 4.22 (s, 2H), 4.69 (s, 4H), 6.45 (s, 1H), 5.58 (s, 1H), 6.82 (d, 4H), 7.13 (d, 4H), 8.15 (d, 1H).

### Example 3

### Preparation of (2S,3R)-3-((2-Aminopyridin-4-yl)methyl)-1-(((R)-1-cyclohexylethyl)carbamoyl)-4-oxoazetidine-2-carboxylic acid hydrochloride (Compound 1.HCl)

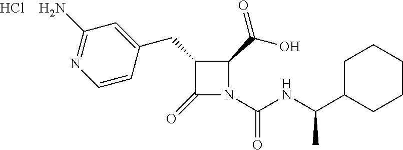 [SMILES: Cl ; C[C@@H](NC(=O)N1C(=O)[C@H](Cc2ccnc(N)c2)[C@H]1C(=O)O)C1CCCCC1]

### Step 1. Preparation of (2S,3R)-3-((2-(bis(4-methoxybenzyl)amino)pyridin-4-yl)methyl)-4-oxoazetidine-2-carboxylic acid

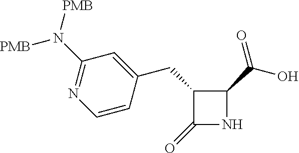

(S)-1-(tert-butyldimethylsilyl)-4-oxoazetidine-2-carboxylic acid was dissolved in THF and cooled to −20° C. The lactam was deprotonated with LDA in THF at about −10 to −20° C. and treated with 4-(bromomethyl)-N,N-bis(4-methoxybenzyl)pyridin-2-amine while maintaining the reaction temperature below −10° C. The reaction was stirred for several hours at −15° C. and then allowed to warm to RT and stirred several hours longer. The mixture was quenched with water and then refluxed for 3 h. The reaction was cooled to RT and treated with 5% aqueous tri-potassium phosphate solution. The phases were separated and the aqueous layer was extracted with EA to remove impurities. The aqueous phase was acidified to pH 3.1 with 6 N HCl and was extracted with EA. This organic phase was dried and concentrated. Residual EA was chased with heptane to produce a slurry which was cooled and filtered. The filter cake was taken up in 40 volumes of IPA and refluxed about 1 h. The mixture was cooled to RT and undissolved solid impurities were removed by filtration. The IPA filtrate was solvent exchanged with heptane, causing the product to precipitate. The slurry was chilled to 5-10° C. and filtered. The filter cake was dried to afford the title compound in 59% yield.

Below is an exemplary procedure of Example 3, Step 1.

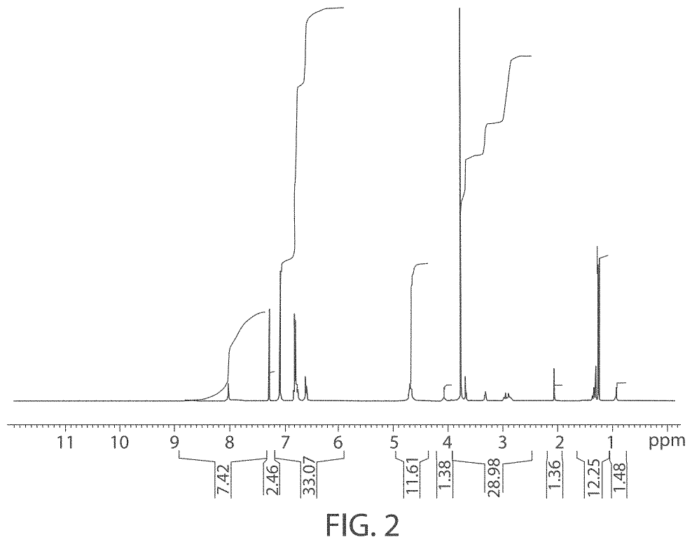

To a solution of (4S)—N-(tert-butyldimethylsilyl))-4-oxoazetidine-2-carboxylic acid (1.30 kg, 5.67 mol) in anhydrous THF (20.8 L) at −20° C. was added LDA (2 M in THF, 6.06 L, 12.13 mol) followed by a solution of 4-(bromomethyl)-N,N-bis(4-methoxybenzyl)pyridine-2-amine (2765.9 g, 6.47 mol) in THF (10.4 L). The resulting mixture was agitated at −20° C. for 5 h then warmed slowly to RT over 16 h. The reaction was monitored by HPLC. Water (2.6 L) was added and the mixture was heated to 60° C. and agitated for 3 h. HPLC analysis indicated the starting material had been consumed. The mixture was cooled to RT and treated with 5% aqueous tri-potassium phosphate solution (38.0 L). The phases were separated, and the aqueous layer was extracted with EA (3×19.5 L). The aqueous layer was acidified to pH 3.1 with 6 N HCl (50 mL) and was extracted with EA (2×39.0 L). The organic phase was dried over MgSO4 and concentrated. Residual EA was chased with heptane (2×2.6 L) to produce a slurry which was filtered, rinsed with heptane (2.6 L), and concentrated. IPA (39.0 L) was added to the solid, and the mixture was refluxed for 1 h. The mixture was cooled to RT, filtered, rinsed with IPA (2.6 L), and the filtrate was concentrated. Heptane (18.2 L) was added to the concentrated solution which caused the product to precipitate from the solution. The solid precipitate was filtered, rinsed with heptane (3.9 L), and dried to afford (2S,3R)-3-((2-(bis(4-methoxybenzyl)amino)pyridin-4-yl)methyl)-4-oxoazetidine-2-carboxylic acid. Exemplary yields and purity are given in Table 2. NMR spectrum of Sample #8 dissolved in CDCl3 is shown in FIG. 2.

TABLE 2
Yields and purity of (2S,3R)-3-((2-(bis(4-methoxybenzyl)amino)
pyridin-4-yl)methyl)-4-oxoazetidine-2-carboxylic acid

| | | | | Purity (%) |
|---|---|---|---|---|
| Sample # | Charge, kg | Yield, g | Yield, % | LC Method 1 |
| 8 | 1.3 | 934.9 | 35.7 | 91.5 |
| 9 | 1.3 | 936.4 | 37.7 | 90.3 |

### Step 2. Preparation of (2S,3R)-3-((2-(bis(4-methoxybenzyl)amino)pyridin-4-yl)methyl)-1-(((R)-1-cyclohexylethyl)carbamoyl)-4-oxoazetidine-2-carboxylic acid

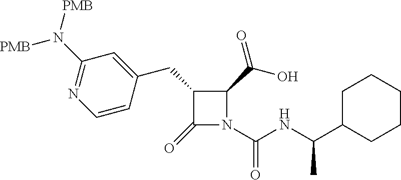

(2S,3R)-3-((2-(bis(4-methoxybenzyl)amino)pyridin-4-ylmethyl)-4-oxoazetidine-2-carboxylic acid was dissolved in DCM and treated with DBU followed by (R)-(1-isocyanatoethyl)cyclohexane (prepared in Example 1) at ambient temperature. After stirring the reaction mixture for several hours, more (R)-(1-isocyanatoethyl)cyclohexane was added and stirred several hours longer. The precipitate that had formed was filtered. The filter cake was rinsed with several portions of 10% aqueous citric acid until no DBU was detected in the DCM phase as determined by HPLC. The DCM phase was dried and concentrated to afford the title compound in 100% yield.

Below is an exemplary procedure of Example 3, Step 2.

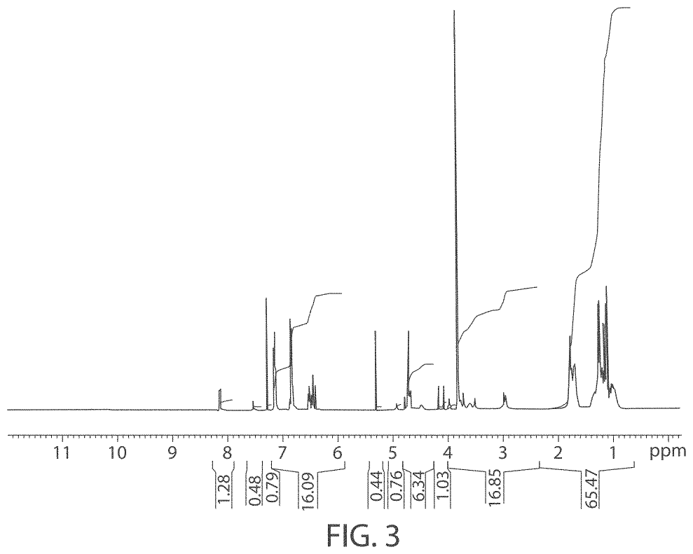

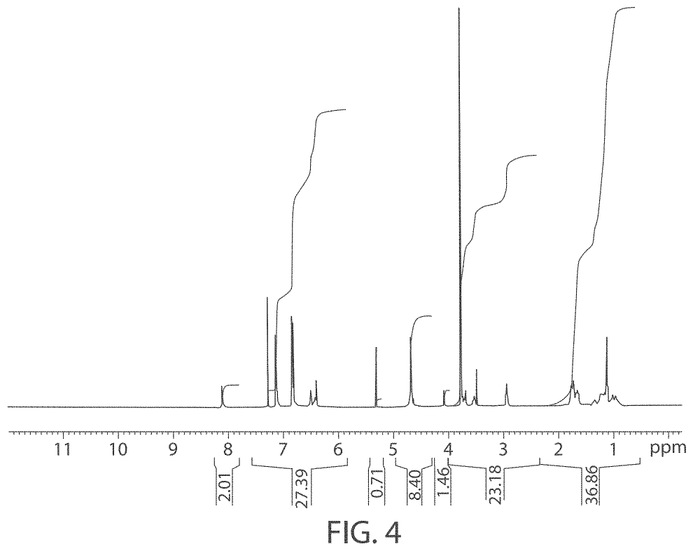

A solution of (2S,3R)-3-((2-(bis(4-methoxybenzyl)amino)pyridin-4-yl)methyl)-4-oxoazetidine-2-carboxylic acid (1868.0 g, 4.55 mol) in DCM (9.34 L) was cooled to 10° C. and treated with DBU (2156.7 g, 15.93 mol) followed by (R)-(1-isocyanatoethyl)cyclohexane (1240.3 g, 8.09 mol) while maintaining the reaction temperature. The reaction mixture was warmed to RT and agitated for 22 h. The reaction was monitored by HPLC. Additional (R)-(1-isocyanatoethyl)cyclohexane (620.2 g, 4.04 g) was added and the mixture was agitated at RT. After 4 h, HPLC analysis indicated the starting material had been consumed. The precipitate that had formed was filtered and washed with DCM (1.9 L). The filtrate was extracted with 10% aqueous citric acid (3×9.34 L). The organic layer was dried over Na₂SO₄, rinsed with DCM (0.5 L), and concentrated to give the crude product. The crude product was chromatographed to afford (2S,3R)-3-((2-(bis(4-methoxybenzyl)amino)pyridin-4-yl)methyl)-1-(((R)-1-cyclohexylethyl)carbamoyl)-4-oxoazetidine-2-carboxylic acid. Exemplary yields and purity are given in Table 3. NMR spectra of the crude and purified product dissolved in CDCl₃ are shown in FIG. 3 and FIG. 4, respectively.

TABLE 3
Yields and purity of (2S,3R)-3-((2-
(bis(4-methoxybenzyl)amino)pyridin-4-yl)methyl)-1-(((R)-
1-cyclohexylethyl)carbamoyl)-4-oxoazetidine-2-carboxylic acid

| | | | | Purity (%) |
|---|---|---|---|---|
| Product | Charge, kg | Yield, g | Yield, % | LC Method 1 |
| Crude | 1.868 | 3177.9 | 127.7 | 46.4 |
| Purified | 3.15 | 1304.8 | 52.2 | 92.9 |

### Step 3. Preparation of (2S,3R)-3-((2-aminopyridin-4-yl)methyl)-1-(((R)-1-cyclohexylethyl)carbamoyl)-4-oxoazetidine-2-carboxylic acid trifluoroacetate

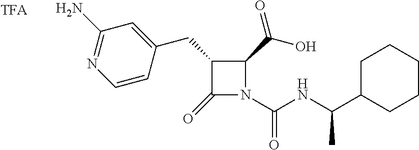 [SMILES: C[C@@H](NC(=O)N1C(=O)[C@H](Cc2ccnc(N)c2)[C@H]1C(=O)O)C1CCCCC1]

(2S,3R)-3-((2-(Bis(4-methoxybenzyl)amino)pyridin-4-yl)methyl)-1-(((R)-1-cyclohexylethyl)carbamoyl)-4-oxoazetidine-2-carboxylic acid was dissolved in TFA and treated with triethylsilane at RT. After stirring several hours, the reaction was concentrated to give a residue. The residue was dissolved in ACN and extracted with hexanes. The mixture was again concentrated to give a residue. The residue was dissolved in DCM and extracted twice with brine. The organic phase was concentrated to afford the title compound in 100% yield.

Below is an exemplary procedure of Example 3, Step 3.

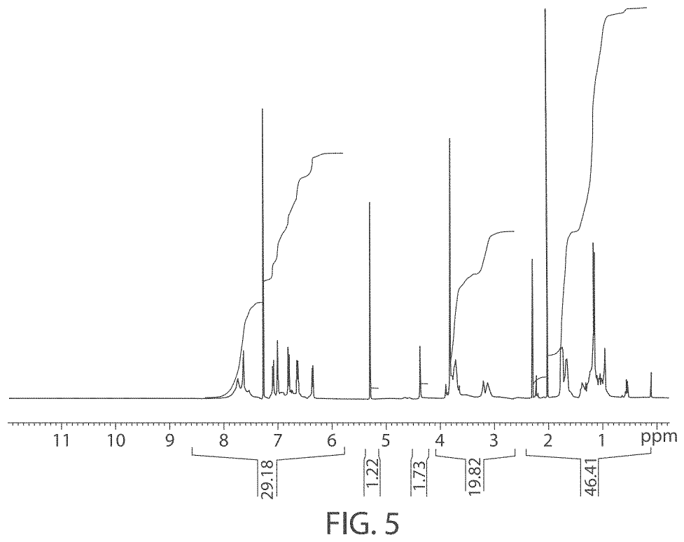

(2S,3R)-3-((2-(Bis(4-methoxybenzyl)amino)pyridin-4-yl)methyl)-1-(((R)-1-cyclohexylethyl)carbamoyl)-4-oxoazetidine-2-carboxylic acid (1.3 kg, 4.55 mol) was added to TFA (13.0 L) at 0° C. To the solution, triethylsilane (0.74 kg, 6.36 mol) was added while maintaining the reaction temperature. The reaction mixture was warmed to RT and agitated for 24 h. The reaction was monitored by HPLC. Additional triethylsilane (0.25 kg, 2.15 mol) was added and the reaction mixture was agitated. After 4 h, HPLC analysis indicated the starting material had been consumed. The mixture was concentrated to give a residue. The residue was dissolved in ACN (13.0 L) and extracted with hexanes (4×13.0 L). The ACN layer was concentrated to give a residue. The residue was dissolved in DCM (13.0 L) and extracted with 13% NaCl solution (2×13.0 L). The organic layer was dried over Na2SO4 and concentrated to afford (2S,3R)-3-((2-aminopyridin-4-yl)methyl)-1-(((R)-1-cyclohexylethyl)carbamoyl)-4-oxoazetidine-2-carboxylic acid trifluoroacetate. Exemplary yield and purity are given in Table 4. NMR spectrum of the product dissolved in CDCl3 is shown in FIG. 5.

TABLE 4
Yield and purity of (2S,3R)-3-((2-
aminopyridin-4-yl)methyl)-1-(((R)-1-cyclohexylethyl)carbamoyl)-
4-oxoazetidine-2-carboxylic acid trifluoroacetate

| | | | Purity (%) |
|---|---|---|---|
| Charge, kg | Yield, g | Yield, % | LC Method 1 |
| 1.3 | 1215.8 | 117.7 | 64.6 |

### Step 4. Preparation of (2S,3R)-3-((2-Aminopyridin-4-yl)methyl)-1-(((R)-1-cyclohexylethyl)carbamoyl)-4-oxoazetidine-2-carboxylic acid hydrochloride (Compound 1.HCl)

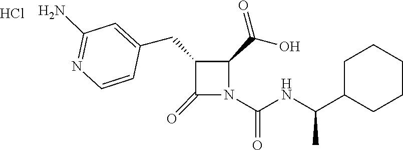 [SMILES: Cl ; C[C@@H](NC(=O)N1C(=O)[C@H](Cc2ccnc(N)c2)[C@H]1C(=O)O)C1CCCCC1]

Below is an exemplary procedure of Example 3, Step 4.

(2S,3R)-3-((2-Aminopyridin-4-yl)methyl)-1-(((R)-1-cyclohexylethyl)carbamoyl)-4-oxoazetidine-2-carboxylic acid trifluoroacetate was dissolved in ACN at RT and treated with 1 M ethereal HCl. After stirring for several hours, seed crystals of the product were added and the mixture was chilled to 0° C. The crystalline product was collected by filtration and dried to give the title compound as a white solid in 55% yield.

Below is an exemplary procedure of Example 3, Step 4.

(2S,3R)-3-((2-Aminopyridin-4-yl)methyl)-1-(((R)-1-cyclohexylethyl)carbamoyl)-4-oxoazetidine-2-carboxylic acid trifluoroacetate (1.03 kg, 3.28 mol) dissolved in ACN (4.84 L) was filtered through a Celite® pad, treated with HCl solution (1M in diethyl ether, 8.43 L, 13.10 mol) and agitated at RT for 42 h. The precipitate that had formed was filtered and washed with diethyl ether (3×0.26 L). The solid was collected and dried in the oven. Exemplary yield and purity are given in Table 5.

TABLE 5
Yield and purity of (2S,3R)-3-
((2-Aminopyridin-4-yl)methyl)-1-(((R)-1-cyclohexylethyl)
carbamoyl)-4-oxoazetidine-2-carboxylic acid hydrochloride

| | | | Purity (%) |
|---|---|---|---|
| Charge, kg | Yield, Kg | Yield, % | LC Method 2 |
| 1.03 | 0.655 | 75.2 | 81.2 |

### Step 5. Purification of (2S,3R)-3-((2-Aminopyridin-4-yl)methyl)-1-(((R)-1-cyclohexylethyl)carbamoyl)-4-oxoazetidine-2-carboxylic acid hydrochloride (Compound 1.HCl)

Below is an exemplary procedure of Example 3, Step 5.

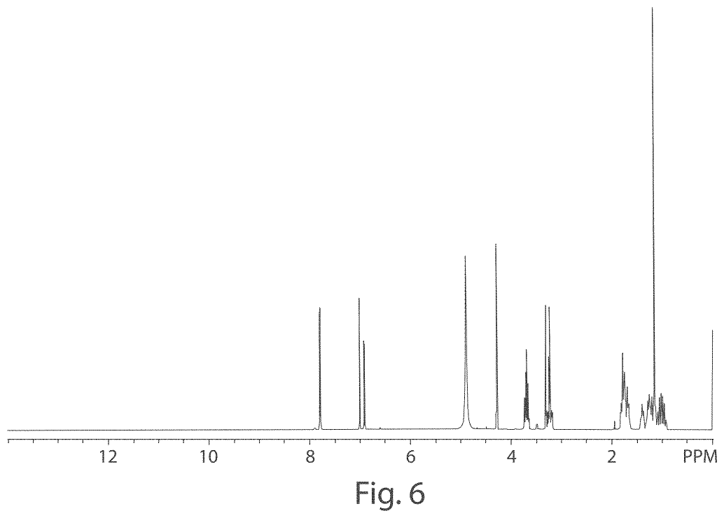

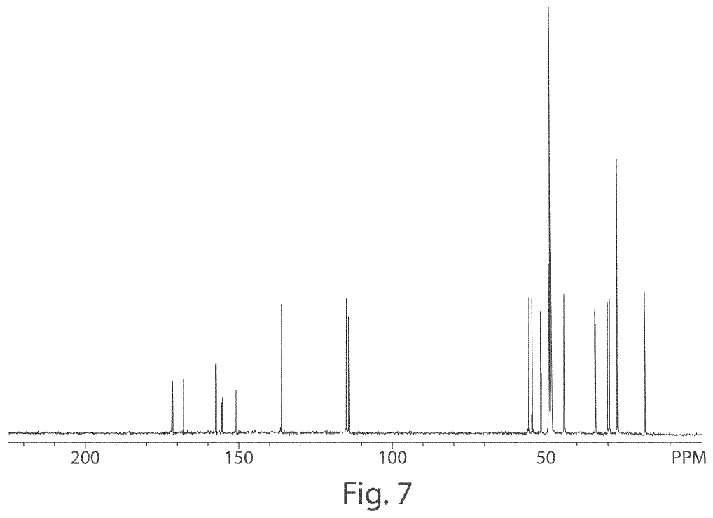

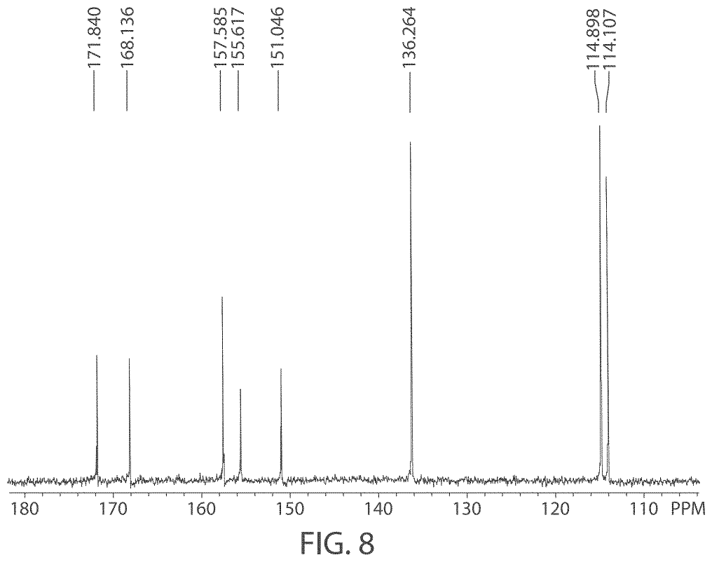

The crude Compound 1.HCl was agitated at RT in IPA (6.3 L) until dissolution. MTBE (6.3 L) was added to the solution and the mixture was agitated for 10 h. The precipitate that had formed was filtered and rinsed with MTBE (2.89 L, 2×1.26 L). The solid was redissolved in IPA (4.8 L), and MTBE (2.4 L) was added drop-wise to the solution and agitated for 131 h. The precipitate that had formed was filtered, rinsed with MTBE (0.96 L, 2×0.64 L), and dried until constant weight. Exemplary yields and purity are given in Table 6. The solid was further triturated with MTBE (1.47 L), filtered, rinsed with MTBE (3×0.74 L) and dried in the oven to constant weight to afford Compound 1.HCl. Exemplary yield and purity are given in Table 7. 1H NMR spectrum of the title compound dissolved in CD3OD is depicted in FIG. 6, and 13C NMR spectrum of the title compound dissolved in CD3OD is depicted in FIG. 7 (blow-up of 13C NMR spectrum in the 105-180 ppm region is depicted in FIG. 8).

TABLE 6
Exemplary yields and purity for the purification of (2S,3R)-3-((2-
Aminopyridin-4-yl)methyl)-1-(((R)-1-cyclohexylethyl)carbamoyl)-
4-oxoazetidine-2-carboxylic acid hydrochloride

| | | | Purity (%) |
|---|---|---|---|
| Charge, kg | Yield, g | Yield, % | LC Method 2 |
| 0.63 | 358.9 | 57.0 | 98 |
| 0.32 | 10.3 | 1.6 | 98 |

TABLE 7
Exemplary yields and purity for the purification of Compound 1•HCl

| | | | Purity (%) |
|---|---|---|---|
| Charge, kg | Yield, g | Yield, % | LC Method 2 |
| 0.63 | 368.4 | 99.9 | 98 |

The purified sample was further analyzed with X-ray powder diffraction (XRPD), the diffractogram for which is shown in FIG. 9. The XRPD pattern was collected with a PANalytical X'Pert PRO MPD diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-run-thick films and analyzed in transmission geometry. A beam-stop, short antiscatter extension, and an antiscatter knife edge were used to minimize the background generated by air. Soller slits for the incident and diffracted beams were used to minimize broadening from axial divergence. The diffraction pattern was collected using a scanning position-sensitive detector (X'Celerator) located 240 mm from the specimen and Data Collector software v. 2.2b. Data acquisition parameters for the pattern are displayed above the image in the Data section of this report including the divergence slit (DS) before the mirror.

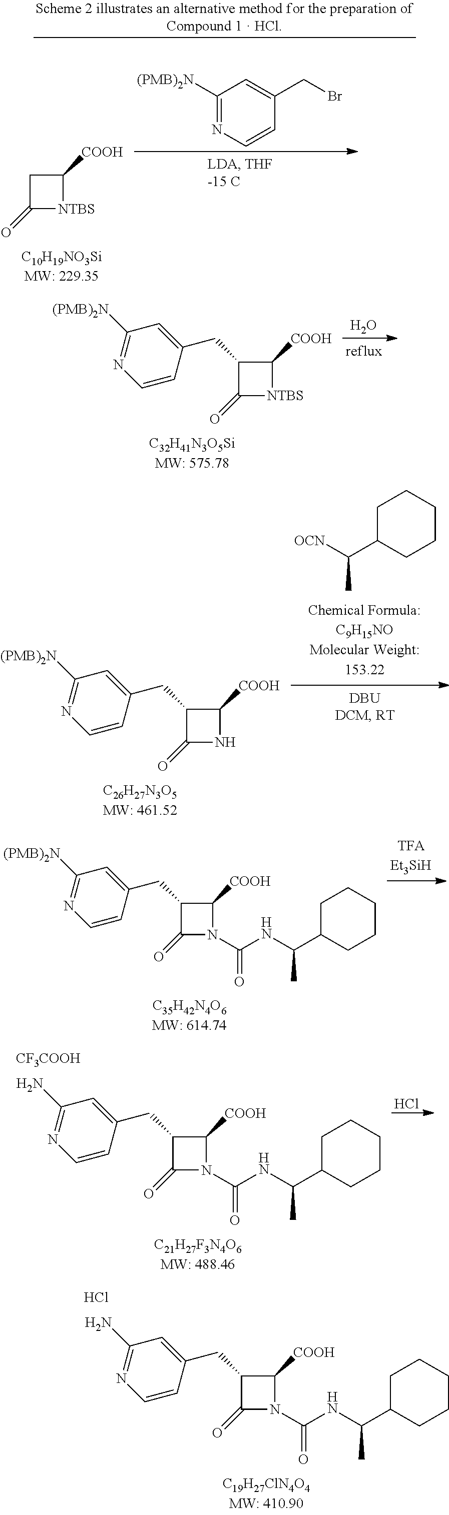

### Example 4

### Synthesis of (2S,3R)-3-((2-aminopyridin-4-yl)methyl)-1-(((R)-1-cyclohexylethyl)carbamoyl)-4-oxoazetidine-2-carboxylic acid hydrochloride

This example describes a synthesis of (2S,3R)-3-((2-aminopyridin-4-yl)methyl)-1-(((R)-1-cyclohexylethyl)carbamoyl)-4-oxoazetidine-2-carboxylic acid hydrochloride in addition to the examples described above.

### Step 1 (2S,3R)-3-((2-(bis(4-methoxybenzyl)amino)pyridin-4-yl)methyl)-4-oxoazetidine-2-carboxylic acid

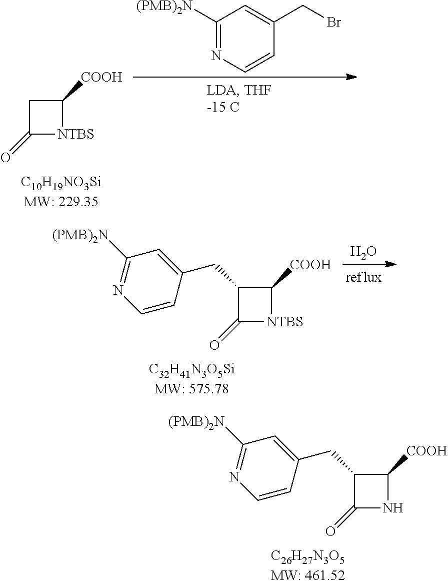

Commercial (S)-1-(tert-butyldimethylsilyl)-4-oxoazetidine-2-carboxylic acid (56.5 g, 0.246 mol) was dissolved in THF (850 mL) and chilled to −40° C. The lactam was deprotonated with lithium diisopropylamide (252.5 mL, 0.505 mol, 2M in THF) at about −40 to −20° C. The resulting mixture was stirred at −40±5° C. for 1 h, cooled to −60±5° C. and then treated with a pre-cooled solution of 4-(bromomethyl)-N,N-bis(4-methoxybenzyl)pyridin-2-amine (100 g, 0.234 mol) in THF (450 mL) while maintaining the temperature below −40° C. The reaction was stirred for several hours at −40±5° C. and allowed to warm to RT and stirred overnight. Then the mixture was quenched with water (565 mL) and heated to 60±5° C. for 1 h. The reaction mixture was cooled to RT and THF was removed at reduced pressure. The aqueous phase was extracted with EA (565 mL×3) to remove impurities. The aqueous phase was acidified to pH 3.1-3.3 with 6 N HCl aqueous solution and extracted with EA (850 mL and then 565 mL). This combined organic phase was dried and concentrated to afford (2S,3R)-3-((2-(bis(4-methoxybenzyl)amino)pyridin-4-yl)methyl)-4-oxoazetidine-2-carboxylic acid (84.1 g) in 74% yield with purity more than 95% (LC method 1).

### Step 2 (2S,3R)-3-((2-(bis(4-methoxybenzyl)amino)pyridin-4-yl)methyl)-1-(((R)-1-cyclohexylethyl)carbamoyl)-4-oxoazetidine-2-carboxylic acid

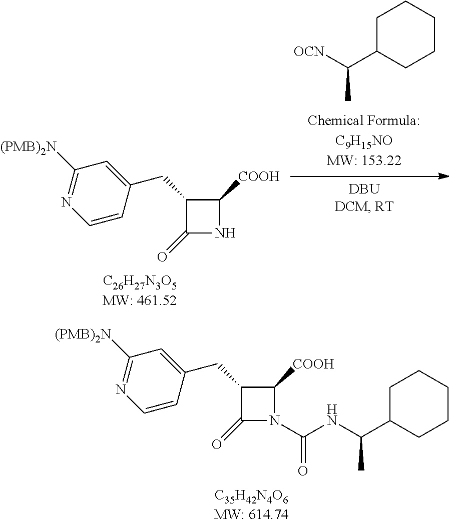

(2S,3R)-3-((2-(Bis(4-methoxybenzyl)amino)pyridin-4-yl)methyl)-4-oxoazetidine-2-carboxylic acid (84 g, 0.182 mol) was dissolved in DCM (210 mL) and treated with DBU (97 g, 0.637 mol) followed by (R)-(1-isocyanatoethyl)cyclohexane (55.8 g, 0.364 mol) at RT. The reaction mixture was stirred at RT overnight. The mixture was diluted with heptane (2500 mL), and stirred at RT for at least 30 min, then it was filtered and dried under vacuum. The crude material was re-slurred in heptane (1000 mL) at RT for at least 4 h. The solids were filtered and dried to give a crude product. The crude product was dissolved in DCM (2250 mL) and washed with 10% aqueous citric acid (1000 mL×3). The DCM phase was dried and concentrated to dryness to afford (2S,3R)-3-((2-(bis(4-methoxybenzyl)amino)pyridin-4-yl)methyl)-1-(((R)-1-cyclohexylethyl)carbamoyl)-4-oxoazetidine-2-carboxylic acid (106 g) in 94.7% yield and a purity of more than 95% (LC method 1).

### Step 3 (2S,3R)-3-((2-aminopyridin-4-yl)methyl)-1-(((R)-1-cyclohexylethyl)carbamoyl)-4-oxoazetidine-2-carboxylic acid-trifluoroacetate

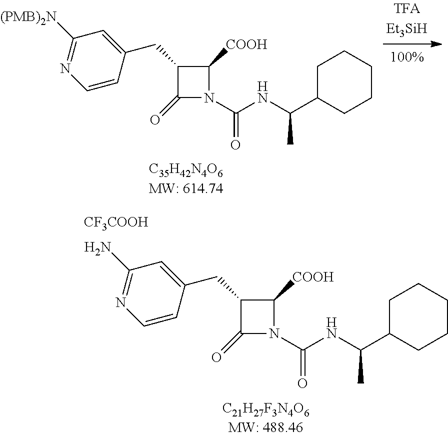

(2S,3R)-3-((2-(Bis(4-methoxybenzyl)amino)pyridin-4-yl)methyl)-1-(((R)-1-cyclohexylethyl)carbamoyl)-4-oxoazetidine-2-carboxylic acid (100 g, 0.163 mol) was dissolved in trifluoroacetic acid (500 mL)) and treated with triethylsilane (75.7 g, 0.65 mol) at RT. After stirring overnight at RT, the reaction mixture was concentrated to give a residue. The residue was dissolved in ACN and extracted with hexanes (3×500 mL). The mixture (ACN layer) was again concentrated to dryness (dried under high vacuum for at least 4 h) to afford (2S,3R)-3-((2-aminopyridin-4-yl)methyl)-1-(((R)-1-cyclohexylethyl)carbamoyl)-4-oxoazetidine-2-carboxylic acid in 100% yield as its trifluoroacetate salt.

### Step 4 (2S,3R)-3-((2-aminopyridin-4-yl)methyl)-1-(((R)-1-cyclohexylethyl)carbamoyl)-4-oxoazetidine-2-carboxylic acid hydrochloride

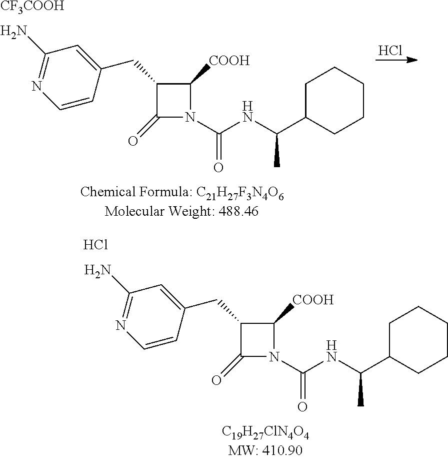 [SMILES: "CF3COOH" ; C[C@@H](NC(=O)N1C(=O)[C@H](Cc2ccnc(N)c2)[C@H]1C(=O)O)C1CCCCC1 ; Cl ; Cl ; C[C@@H](NC(=O)N1C(=O)[C@H](Cc2ccnc(N)c2)[C@H]1C(=O)O)C1CCCCC1]

(2S,3R)-3-((2-Aminopyridin-4-yl)methyl)-1-(((R)-1-cyclohexylethyl)carbamoyl)-4-oxoazetidine-2-carboxylic acid trifluoroacetate was dissolved in ACN (350 mL) at RT. The turbid solution was filtered and the insolubles were rinsed with ACN (50 mL). The filtrate was cooled to 5±5° C. and treated with hydrochloric acid (488 mL, 1 N ethereal solution). The mixture was stirred at RT overnight. The crystalline product was collected by filtration and dried. The crude product was slurried in isopropyl alcohol (100 mL) at RT overnight, filtered, and the solids were rinsed MTBE (2×50 mL) to give (2S,3R)-3-((2-aminopyridin-4-yl)methyl)-1-(((R)-1-cyclohexylethyl)carbamoyl)-4-oxoazetidine-2-carboxylic acid hydrochloride as a white solid (34.8 g) in 71% yield and 98.1% purity (LC method 2).

### Example 5

### HPLC Procedure for the Analysis of Purity (Area %)

### LC Method 1

Summary: Samples are diluted in the appropriate diluent. The resulting solutions are analyzed using reversed phase HPLC with UV detection performed at 215 nm.

Equipment and Materials:
Agilent 1100/1200 HPLC system or equivalent
Column: Agilent Eclipse Plus C18, 4.6×100 mm, 3.5 μm
Trifluoroacetic Acid (TFA), EMD or equivalent
HPLC grade Acetonitrile (ACN), Fisher or equivalent
HPLC grade Water (H₂O), Fisher or equivalent
0.45 μm PTFE syringe filter

Solution Preparation:
Mobile Phase A: 0.1% TFA in H₂O
Add 1.0 mL of TFA to 1.0 L of H₂O. Mix well. Scale as needed.
Mobile Phase B: 0.1% TFA in ACN
Add 1.0 mL of TFA to 1.0 L of ACN. Mix well. Scale as needed
Diluent A: 1:1 ACN/H₂O
Combine equal volumes of CAN and Water. Mix well. Scale as needed.
Diluent B: ACN

TABLE 7
Chromatographic conditions for LC method 1

| | |
|---|---|
| Column: | Agilent Eclipse Plus C18, 4.6 × 100 mm, |
| | 3.5 μm |
| Mobile Phase A: | 0.1% TFA in H₂O |
| Mobile Phase B: | 0.1% TFA in ACN |
| Detection: | 215 nm* |
| Column Temperature: | 30° C. |
| Injection Volume: | 5.0 μL |
| Flow Rate: | 1.0 mL/min |
| Acquisition Time: | 20.0 minutes, plus 5.0 minute post run |

*If DAD is used, Ref. = off, Bw = 8 nm, Slit = 16 nm.

TABLE 8
Gradient for LC method 1

| Time | | |
|---|---|---|
| (minutes) | % A | % B |
| 0.0 | 95 | 5 |
| 15.0 | 0 | 100 |
| 20.0 | 0 | 100 |

+5.0 minute re-equilibration time

### LC Method 2

Summary: Samples are diluted in 1:1 Acetonitrile:Water. The resulting solutions are analyzed using reversed phase HPLC with UV detection performed at 215 nm.

Equipment and Materials:
Agilent 1100/1200 HPLC system or equivalent
Column: Agilent Eclipse Plus C18, 4.6×100 mm, 3.5 μm
Phosphoric Acid, 85% (H₃PO₄), Macron or equivalent
HPLC grade Acetonitrile (CAN), Fisher or equivalent
HPLC grade Water (H₂O), Fisher or equivalent

Solution Preparation:
Mobile Phase A: 0.1% H₃PO₄ in H₂O
Add 1.0 mL of H₃PO₄ to 1.0 L of H₂O. Mix well. Scale as needed.
Mobile Phase B: 0.1% H₃PO₄ in ACN
Add 1.0 mL of H₃PO₄ to 1.0 L of ACN. Mix well. Scale as needed.
Diluent: 1:1 ACN/H₂O
Combine equal volumes of ACN and Water. Mix well. Scale as needed.

TABLE 9
Chromatographic conditions for LC method 2

| | |
|---|---|
| Column: | Agilent Eclipse Plus C18, 4.6 × 100 mm, |
| | 3.5 μm |
| Mobile Phase A: | 0.1% H₃PO₄ in H₂O |
| Mobile Phase B: | 0.1% H₃PO₄ in ACN |
| Detection: | 215 nm* |
| Column Temperature: | 30° C. |
| Injection Volume: | 5.0 μL |
| Flow Rate: | 1.0 mL/min |
| Acquisition Time: | 20.0 minutes, plus 5.0 minute post run |

*If DAD is used, Ref. = off, Bw = 8 nm, Slit = 16 nm.

TABLE 10
Gradient for LC method 2

| Time | | |
|---|---|---|
| (minutes) | % A | % B |
| 0.0 | 95 | 5 |
| 15.0 | 0 | 100 |
| 20.0 | 0 | 100 |

+5.0 minute re-equilibration time

### Example 6

### Efficacy Study of Compound 1 in a Hound Cardiopulmonary Bypass Model

The objective of this study was to demonstrate the efficacy of Compound 1 compared to the Standard of Care (SOC), heparin, for preventing activation of blood coagulation components while using the Cardiopulmonary Bypass (CPB) circuit during an extended run time on Day 1 in a mixed breed hound dog model. The study design is shown in Table 11:

TABLE 11
Experimental Design (Target Doses of Compound 1^{b})

| | | IV | IV Bolus | | IV | Dose | Dose | | |
|---|---|---|---|---|---|---|---|---|---|
| | | Loading | Dose | Dose | Infusion | Infusion | Concentration | | Compound |
| | No. of | Dose | Volume | Concentration | Dose Level | Rate | for IV | | 1 in Prime |
| Group | Animals | (mg/kg) | (mL/kg) | for IV Bolus | (mg/kg/hr) | (mL/kg/hr) | Infusion | | Solution |
| 1 | 3 | NA | NA | NA | 3 | 5 | 0.6 | mg/mL | NA |
| 2 | 2 | 3 | 1 | 0.6 μg/mL and | 3 | 5 | 0.6 | μg/mL | 0.01 mg/mL |
| | | | | 3 mg/mL^{a} | | | | | |
| 3 | 2 | 10 | 1 | 10 mg/mL | 10 | 5 | 2.0 | mg/mL | 0.01 mg/mL |
| 4 | 2 | 10 | 1 | 10 mg/mL | 10 | 5 | 2.0 | mg/mL | 0.01 mg/mL |
| 5 | 2 | 10 | 1 | 10 mg/mL | 10 | 5 | 2.0 | mg/mL | 0.01 mg/mL |

NA—No Applicable
^{a}Animal No. 1001 received 0.6 μg/mL and Animal No. 1004 received 3 mg/mL.
^{b}Doses shown are targets for the dosing on this study; actual dose values are shown in the results section.

The following parameters and endpoints were evaluated in this study: mortality, body weight, physical, clinical pathology parameters (hematology and coagulation), 1212coagulation time, and bioanalytical parameters.

### Experimental Design

### Administration

The vehicle and test article were administered via intravenous (IV) infusion once on Day 1 for 135 minutes (initiated 30 minutes prior to starting the Cardiopulmonary Bypass (CPB) and continuing for 105 minutes of CPB). Group 2 animals received a 0.6 μg/mL or 3.0 mg/mL IV bolus dose immediately prior to the start of IV infusion. Group 3, 4, and 5 animals received a 10 mg/kg IV bolus dose prior to the start of the IV infusion; with the CPB machine primed with test article at 10 μg/mL.

### Surgical Procedure

Group 1 had an infusion pump setup with an open system/reservoir. Infusion of the Compound 1 was started 30 minutes prior to the animal being placed on the CPB pump. The CPB pump was primed with 0.9% saline.

Groups 2, 3, and 4 had an infusion pump setup with an open system/reservoir. Venous and arterial sheaths were flushed with the Compound 1 at a concentration of 10 μg/mL. An IV bolus dose of the test article was administered immediately prior to the start of the infusion. Infusion of Compound 1 was started 30 minutes prior to the animal being placed on the CPB pump. The CPB patient was primed with 10 μg/mL of the Compound 1 prior to initiation of the CPB pump.

Group 5 had an infusion pump setup with a closed system/“bag.” Venous and arterial sheaths were then flushed with Compound 1 at 10 μg/mL. An IV bolus dose of the Compound 1 was administered immediately prior to the start of the infusion. Infusion of Compound 1 was started 30 minutes prior to the animal being placed on the CPB pump.

### Results

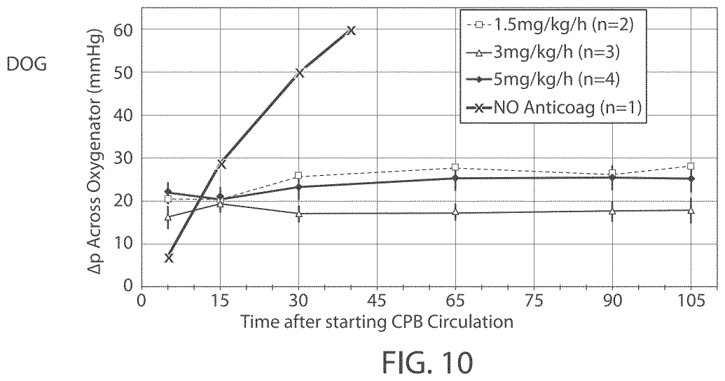

FIG. 10 shows pressure gradients assessed across the membrane oxygenator. Studies previously conducted with no anticoagulant demonstrated that the pressure across the membrane oxygenator built within 15 minutes of pump start and exponentially increased over the next 30 minutes such that the oxygenator was occluded and the circulation was stopped, whereas with Compound 1 at multiple doses, the pressure gradient across the membrane oxygenator stayed consistent through the entire run, indicating that the test article successfully maintained anticoagulation allowing the continuation of the pump run for the entirety of the protocol.

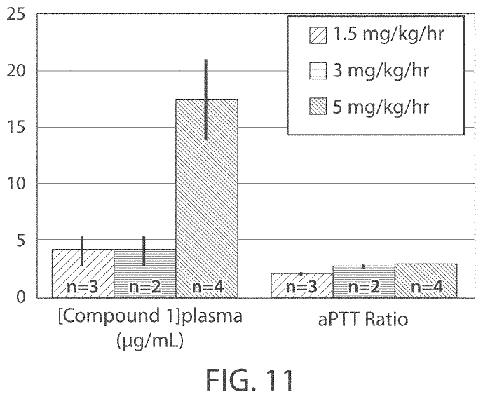

FIG. 11 shows a correlation between Compound 1 plasma concentration and aPTT. All animals survived to study termination. Overall, Compound 1 was not associated with any increases in morbidity or mortality at the dose levels used in this study during the Cardiopulmonary bypass/ECMO protocol.

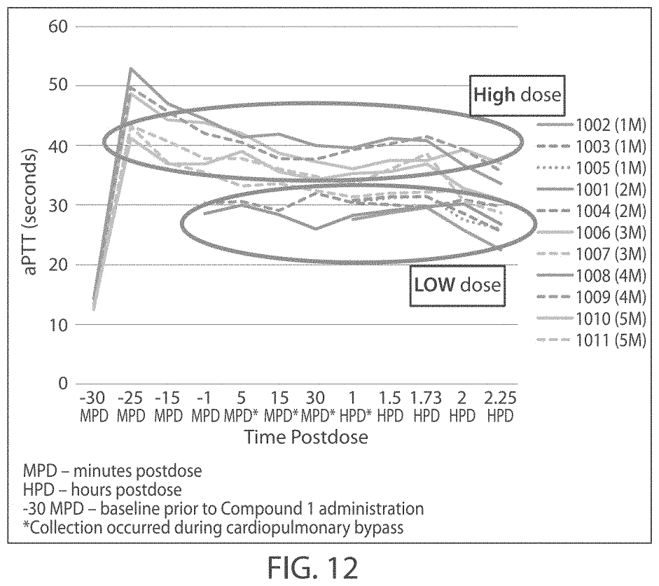

During Compound 1 infusion and prior to CPB, aPTT was moderately to markedly prolonged in all animals (FIG. 12). Prolongations in aPTT persisted throughout Compound 1 infusion and CPB. In groups that received a loading dose of Compound 1 (Groups 2 through 5), prolongations in aPTT were most pronounced prior to (Group 3 through 5) or during the first 30 minutes of CPB (Group 2), but then improved slightly before reaching steady-state. Group 1 animals did not receive an Compound 1 loading dose, and prolongations in aPTT remained relatively consistent at all measured timepoints during Compound 1 infusion in this group. In all groups following cessation of Compound 1 infusion and CPB, aPTT trended towards baseline values, but remained moderately prolonged at the conclusion of the study.

### Conclusions

Administration of the Compound 1 to the model was successful in preventing the activation of blood coagulation in components of cardiopulmonary bypass. The anticoagulant effects of Compound 1 were selective to inhibition of activated partial thromboplastin time (aPTT). Additionally, the data demonstrated that adding a bolus dose immediately prior to starting the infusion enabled targeted plasma levels of Compound 1 to rapidly be achieved, along with desired steady state levels, and was sufficient to achieve a successful 105-minute CPB run and prevent coagulation in most of the circuit components.

Overall, these data indicate that Compound 1 may be an acceptable alternative to heparin in preventing blood coagulation in components of cardiopulmonary bypass.

### Equivalents

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure

What is claimed is:

1. A process for preparing a pharmaceutically acceptable salt of Formula (I) 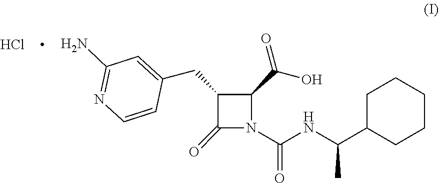 [SMILES: Cl ; C[C@@H](NC(=O)N1C(=O)[C@H](Cc2ccnc(N)c2)[C@H]1C(=O)O)C1CCCCC1]
or a solvate (e.g., a hydrate) thereof, comprising dissolving a salt of Formula (II) 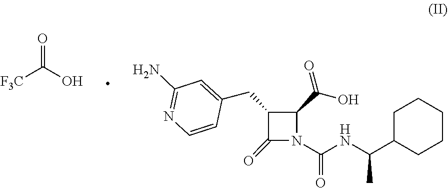 [SMILES: O=C(O)C(F)(F)F ; C[C@@H](NC(=O)N1C(=O)[C@H](Cc2ccnc(N)c2)[C@H]1C(=O)O)C1CCCCC1]
or a solvate (e.g., a hydrate) thereof in a solvent, thereby preparing a first solution, and adding hydrogen chloride to the first solution,
thereby producing the pharmaceutically acceptable salt of Formula (I).

2. The process of claim 1, wherein the salt of Formula (II) is dissolved in an aprotic solvent.

3. The process of claim 1 or 2, wherein the solvent comprises (e.g., consists of or consists essentially of) acetonitrile.

4. The process of any one of claims 1 to 3, wherein the hydrogen chloride is added to the first solution by bubbling HCl gas into the first solution or by adding a second solution comprising HCl (e.g., an ethereal hydrochloric acid solution) to the first solution.

5. The process of any one of claims 1 to 4, wherein the starting quantity of the salt of Formula (II) or solvate (e.g., a hydrate) thereof is greater than or equal to 500 grams.

6. The process of any one of claims 1 to 5, wherein the starting quantity of the salt of Formula (II) or solvate (e.g., a hydrate) thereof is greater than or equal to 1 kilogram.

7. The process of any one of claims 1 to 6, wherein the process produces over 300 grams (e.g., over about 350 grams (e.g., about 368 grams)) of pharmaceutically acceptable salt of Formula (I) or solvate (e.g., a hydrate) thereof.

8. The process of any one of claims 1 to 7, wherein the process produces the pharmaceutically acceptable salt of Formula (I) or solvate (e.g., a hydrate) thereof in a yield greater than about 50% (e.g., in about 55% yield).

9. The process of any one of claims 1 to 8, wherein the process produces the pharmaceutically acceptable salt of Formula (I) or solvate (e.g., a hydrate) thereof in a yield greater than about 75%.

10. The process of any one of claims 1 to 9, wherein the process produces the pharmaceutically acceptable salt of Formula (I) or solvate (e.g., a hydrate) thereof in a yield greater than about 90%.

11. The process of any one of claims 1 to 10, wherein the process produces the pharmaceutically acceptable salt of Formula (I) or solvate (e.g., a hydrate) thereof in a yield greater than about 99%.

12. The process of any one of claims 1 to 11, wherein the purity of the pharmaceutically acceptable salt of Formula (I) or solvate (e.g., a hydrate) thereof is about 80%.

13. The process of any one of claims 1 to 12, wherein the purity of the pharmaceutically acceptable salt of Formula (I) or solvate (e.g., a hydrate) thereof is about 81%.

14. The process of any one of claims 1 to 13, further comprising purifying the pharmaceutically acceptable salt of Formula (I) or solvate (e.g., a hydrate) thereof by dissolving the pharmaceutically acceptable salt of Formula (I) or solvate (e.g., a hydrate) thereof in a solvent (e.g., isopropyl alcohol) followed by precipitation of the dissolved pharmaceutically acceptable salt of Formula (I) or solvate (e.g., a hydrate) thereof using another solvent (e.g., methyl tert-butyl ether).

15. The process of claim 14, wherein the purity of the pharmaceutically acceptable salt of Formula (I) or solvate (e.g., a hydrate) thereof after precipitation is greater than 98%.

16. The process of claim 14 or 15, wherein the purity of the pharmaceutically acceptable salt of Formula (I) or solvate (e.g., a hydrate) thereof after precipitation is about 98%.

17. The process of any one of claims 1 to 13, further comprising purifying the pharmaceutically acceptable salt of Formula (I) or solvate (e.g., a hydrate) thereof by slurrying the pharmaceutically acceptable salt of Formula (I) or solvate (e.g., a hydrate) thereof in a solvent (e.g., isopropyl alcohol) and then filtering the pharmaceutically acceptable salt of Formula (I) or solvate (e.g., a hydrate) thereof to separate the pharmaceutically acceptable salt of Formula (I) or solvate (e.g., a hydrate) thereof from the solvent.

18. The process of claim 17, wherein the purity of the pharmaceutically acceptable salt of Formula (I) or solvate (e.g., a hydrate) thereof after slurrying and separating is greater than 98%.

19. The process of claim 17 or 18, wherein the purity of the pharmaceutically acceptable salt of Formula (I) or solvate (e.g., a hydrate) thereof after slurrying and separating is about 98%.

20. The process of any one of claims 1 to 19, comprising preparing the salt of Formula (II) by contacting a compound of Formula (III) 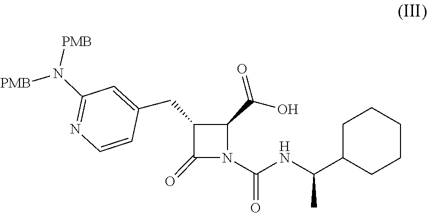
with trifluoroacetic acid.

21. The process of any one of claims 1 to 20, further comprising contacting the compound of Formula (III) with a silane (e.g., triethylsilane).

22. The process of any one of claims 1 to 21, wherein the process produces over 500 grams of the compound of Formula (III) (e.g., over 1 kg).

23. The process of any one of claims 1 to 22, comprising preparing the compound of Formula (III) by contacting a compound of Formula (IV) 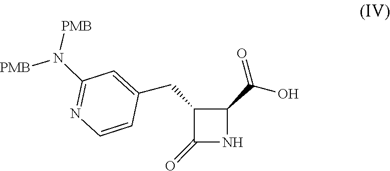
with a compound of Formula (V)

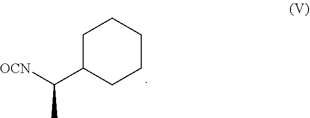

24. The process of any one of claims 1 to 23, wherein the process produces over 1 kilogram of the compound of Formula (III) (e.g., about 1.3 kg).

25. The process of any one of claims 1 to 24, wherein the process is carried out in the presence of a solvent.

26. The process of any one of claims 1 to 25, wherein the process is carried out in the presence of a base (e.g., 1,8-diazabicyclo(5.4.0)undec-7-ene).

27. The process of any one of claims 1 to 26, comprising preparing the compound of Formula (IV) by contacting a compound of Formula (VI) 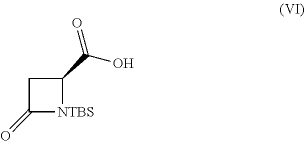
with a compound of Formula (VII)

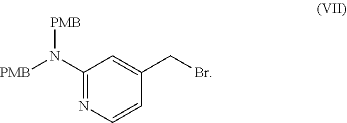

28. The process of any one of claims 1 to 27, wherein the process produces over 500 grams of the compound of Formula (IV) (e.g., over 900 grams).

29. The process of any one of claims 1 to 28, wherein the compound of Formula (III) is purified by a purification method that is not chromatography.

30. The process of claim 29, wherein the purification method comprises slurrying the compound of Formula (III) in a solvent (e.g., heptane) and then filtering the compound of Formula (III) to separate the compound of Formula (III) from the solvent.

31. The process of any one of claims 1 to 30, wherein the purity of the compound of Formula (III) is greater than 90%.

32. The process of any one of claims 1 to 31, wherein the compound of Formula (I) is purified by a purification method that is not chromatography.

33. A crystalline pharmaceutically acceptable salt of the Formula (I):

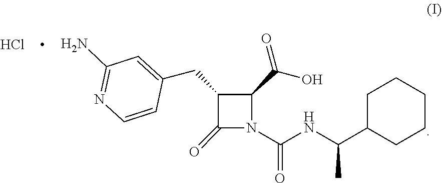 [SMILES: Cl ; C[C@@H](NC(=O)N1C(=O)[C@H](Cc2ccnc(N)c2)[C@H]1C(=O)O)C1CCCCC1]

34. A method of treating a thromboembolic disorder in a subject in need thereof, the method comprising administering to the subject an effective amount of a compound represented by 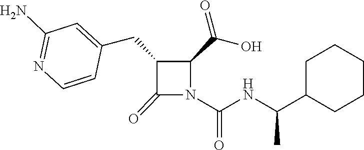 [SMILES: C[C@@H](NC(=O)N1C(=O)[C@H](Cc2ccnc(N)c2)[C@H]1C(=O)O)C1CCCCC1]
or a pharmaceutically acceptable salt thereof, wherein the blood of the subject is contacted with an artificial surface.

35. A method of reducing the risk of a thromboembolic disorder in a subject in need thereof, the method comprising administering to the subject an effective amount of a compound represented by 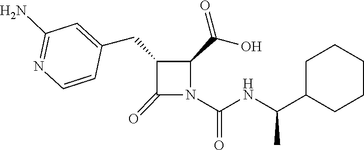 [SMILES: C[C@@H](NC(=O)N1C(=O)[C@H](Cc2ccnc(N)c2)[C@H]1C(=O)O)C1CCCCC1]
or a pharmaceutically acceptable salt thereof, wherein the blood of the subject is contacted with an artificial surface.

36. A method of prophylaxis of a thromboembolic disorder in a subject in need thereof, the method comprising administering to the subject an effective amount of a compound represented by 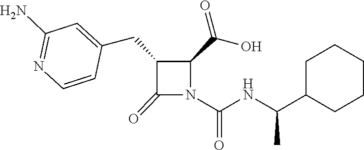 [SMILES: C[C@@H](NC(=O)N1C(=O)[C@H](Cc2ccnc(N)c2)[C@H]1C(=O)O)C1CCCCC1]
or a pharmaceutically acceptable salt thereof, wherein the blood of the subject is contacted with an artificial surface.

37. The method of any one of claims 33 to 36, wherein the artificial surface is in contact with blood in the subject's circulatory system.

38. The method of any one of claims 33 to 37, wherein the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus.

39. The method of any one of claims 33 to 38, wherein the artificial surface causes or is associated with the thromboembolic disorder.

40. The method of any one of claims 33 to 39, wherein the thromboembolic disorder is a venous thromboembolism, deep vein thrombosis, or pulmonary embolism.

41. The method of any one of claims 33 to 39, wherein the thromboembolic disorder is a blood clot.

42. The method of any one of claims 33 to 41, further comprising conditioning the artificial surface with a separate dose of the compound or pharmaceutically acceptable salt thereof, prior to contacting the artificial surface with blood in the circulatory system of the subject.

43. The method of any one of claims 33 to 42, further comprising conditioning the artificial surface with a separate dose of the compound or pharmaceutically acceptable salt thereof prior to or during administration of the compound or a pharmaceutically acceptable salt thereof to the subject.

44. The method of any one of claims 33 to 43, further comprising conditioning the artificial surface with a separate dose of the compound or pharmaceutically acceptable salt thereof prior to and during administration of the compound or a pharmaceutically acceptable salt thereof to the subject.

45. A method of treating the blood of a subject in need thereof, the method comprising administering to the subject an effective amount of a compound represented by 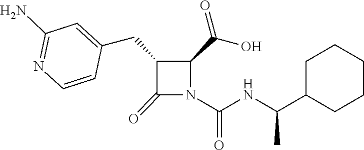 [SMILES: C[C@@H](NC(=O)N1C(=O)[C@H](Cc2ccnc(N)c2)[C@H]1C(=O)O)C1CCCCC1]
or a pharmaceutically acceptable salt thereof.

46. A method of maintaining the plasma level of a compound represented by 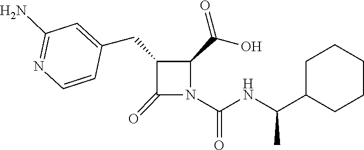 [SMILES: C[C@@H](NC(=O)N1C(=O)[C@H](Cc2ccnc(N)c2)[C@H]1C(=O)O)C1CCCCC1]
or a pharmaceutically acceptable salt thereof, in the blood of a subject in contact with an artificial surface, the method comprising:
(i) administering the compound or pharmaceutically acceptable salt thereof to the subject prior to or while contacting the artificial surface with the blood of the subject; and
(ii) conditioning an artificial surface with the compound or a pharmaceutically acceptable salt thereof prior to or while contacting the artificial surface with the blood of the subject;
thereby maintaining the plasma level of the compound or a pharmaceutically acceptable salt thereof in the blood of the subject.

47. The method of claim 46, wherein the compound, or a pharmaceutically acceptable salt thereof, maintains a constant activated partial thromboplastin time (aPTT) in the blood of the subject before and after contact with the artificial surface.

48. The method of claim 46 or 47, wherein the compound or a pharmaceutically acceptable salt thereof is administered to the subject prior to and while contacting the artificial surface with the blood of the subject.

49. The method of any one of claims 46 to 48, wherein the artificial surface is conditioned with the compound or a pharmaceutically acceptable salt thereof prior to and while contacting the artificial surface with the blood of the subject.

50. The method of any one of claims 46 to 49, wherein the method further prevents or reduces risk of a blood clot formation in the blood of the subject in contact with the artificial surface.

51. The method of any one of claims 48 to 50, wherein the artificial surface is a cardiopulmonary bypass circuit.

52. The method of any one of claims 48 to 50, wherein the artificial surface is an extracorporeal membrane oxygenation (ECMO) apparatus.

53. The method of claim 52, wherein the ECMO apparatus is venovenous ECMO apparatus or venoarterial ECMO apparatus.

54. A method of preventing or reducing a risk of a thromboembolic disorder in a subject during or after a medical procedure, comprising:
(i) administering to the subject an effective amount of a compound represented by: 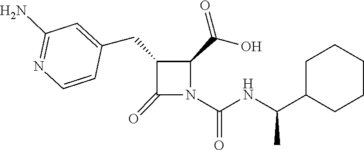 [SMILES: C[C@@H](NC(=O)N1C(=O)[C@H](Cc2ccnc(N)c2)[C@H]1C(=O)O)C1CCCCC1]
or pharmaceutically acceptable salt thereof, before, during, or after the medical procedure; and
(ii) contacting blood of the subject with an artificial surface;
thereby preventing or reducing the risk of the thromboembolic disorder during or after the medical procedure.

55. The method of claim 54, wherein the artificial surface is conditioned with the compound or pharmaceutically acceptable salt thereof prior to administration of the compound to the subject prior to, during, or after the medical procedure.

56. The method of claim 54 or 55, wherein the artificial surface is conditioned with a solution comprising the compound or a pharmaceutically acceptable salt thereof prior to administration of the compound or a pharmaceutically acceptable salt thereof to the subject prior to, during, or after the medical procedure.

57. The method of claim 56, wherein the solution is a saline solution, Ringer's solution, or blood.

58. The method of any one of claims 54 to 57, wherein the thromboembolic disorder is a blood clot.

59. The method of any one of claims 54 to 58, wherein the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

60. The method of any one of claims 54 to 59, wherein the medical procedure comprises a cardiopulmonary bypass.

61. The method of any one of claims 54 to 59, wherein the medical procedure comprises an oxygenation and pumping of blood via extracorporeal membrane oxygenation (ECMO).

62. The method of claim 61, wherein the ECMO is venovenous ECMO or venoarterial ECMO.

63. The method of any one of claims 34 to 62, wherein the pharmaceutically acceptable salt of the compound is a hydrochloride salt.

64. The method of any one of claims 34 to 63, wherein the compound is administered to the subject intravenously.

65. The method of any one of claims 34 to 63, wherein the compound is administered to the subject subcutaneously.

66. The method of any one of claims 34 to 63, wherein the compound is administered to the subject as a continuous intravenous infusion.

67. The method of any one of claims 34 to 63, wherein the compound is administered to the subject as a bolus.

68. The method of any one of claims 34 to 67, wherein the subject is a human.

69. The method of any one of claims 34 to 68, wherein the subject has an elevated risk of a thromboembolic disorder.

70. The method of claim 69, wherein the thromboembolic disorder is a result of a complication in surgery.

71. The method of any one of claims 34 to 70, wherein the subject is sensitive to or has developed sensitivity to heparin.

72. The method of any one of claims 34 to 71, wherein the subject is resistant to or has developed resistance to heparin.

73. The method of any one of claims 34 to 72, wherein the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

74. The method of any one of claims 34 to 72, wherein the subject is a pediatric subject.

75. The method of any one of claims 34 to 72, wherein the subject is an adult.
